SEQUENCE LISTING (70 sequences)

SEQ ID NO: 1 - protein, 20 aa, Artificial Sequence
  misc_feature: Novel Sequence
GGIYYPYDIYYPYDIYYPYD

SEQ ID NO: 2 - protein, 20 aa, Artificial Sequence
  misc_feature: Novel Sequence
GGIYWRYDIYWRYDIYWRYD

SEQ ID NO: 3 - protein, 19 aa, Artificial Sequence
  misc_feature: Novel Sequence
GGYYRYDIYYRYDIYYRYD

SEQ ID NO: 4 - protein, 4 aa, Artificial Sequence
  misc_feature: Novel Sequence
YYPY

SEQ ID NO: 5 - protein, 4 aa, Artificial Sequence
  misc_feature: Novel Sequence
PWLY

SEQ ID NO: 6 - protein, 5 aa, Artificial Sequence
  misc_feature: Novel Sequence
YYPYD

SEQ ID NO: 7 - protein, 5 aa, Artificial Sequence
  misc_feature: Novel Sequence
YYRYD

SEQ ID NO: 8 - protein, 5 aa, Artificial Sequence
  misc_feature: Novel Sequence
YYRGD

SEQ ID NO: 9 - protein, 8 aa, Artificial Sequence
  misc_feature: Novel Sequence
APWLYGPA

SEQ ID NO: 10 - protein, 5 aa, Artificial Sequence
  misc_feature: Novel Sequence
APWLY

SEQ ID NO: 11 - protein, 5 aa, Artificial Sequence
  misc_feature: Novel Sequence
FSLLW

SEQ ID NO: 12 - protein, 7 aa, Artificial Sequence
  misc_feature: Novel Sequence
APWLYAP

SEQ ID NO: 13 - protein, 4 aa, Artificial Sequence
  misc_feature: Novel Sequence
WPYL

SEQ ID NO: 14 - protein, 4 aa, Artificial Sequence
  misc_feature: Novel Sequence
WRDG

SEQ ID NO: 15 - protein, 4 aa, Artificial Sequence
  misc_feature: Novel Sequence
YRGD

SEQ ID NO: 16 - protein, 15 aa, Artificial Sequence
  misc_feature: Novel Sequence
AGVALGSQSYGLHGP

SEQ ID NO: 17 - protein, 15 aa, Artificial Sequence
  misc_feature: Novel Sequence
TWPVVHGACRAHGHC

SEQ ID NO: 18 - protein, 15 aa, Artificial Sequence
  misc_feature: Novel Sequence
GFVLVYTFPSSVCCP

SEQ ID NO: 19 - protein, 15 aa, Artificial Sequence
  misc_feature: Novel Sequence
LAFVWTVAVPPFPPG

SEQ ID NO: 20 - protein, 15 aa, Artificial Sequence
  misc_feature: Novel Sequence
GLDLLGDVRIPVVRR

SEQ ID NO: 21 - protein, 15 aa, Artificial Sequence
  misc_feature: Novel Sequence
GLDLLGDVRIPVVAS

SEQ ID NO: 22 - protein, 15 aa, Artificial Sequence
  misc_feature: Novel Sequence
SLVSSLDIRVFHRLP

SEQ ID NO: 23 - protein, 15 aa, Artificial Sequence
  misc_feature: Novel Sequence
VGITGFVDPLPLRLL

SEQ ID NO: 24 - protein, 15 aa, Artificial Sequence
  misc_feature: Novel Sequence
GAFSSPRSLTVPLRR

SEQ ID NO: 25 - protein, 15 aa, Artificial Sequence
  misc_feature: Novel Sequence
LRASFFLEAARGSAS

SEQ ID NO: 26 - protein, 15 aa, Artificial Sequence
  misc_feature: Novel Sequence
AGRWVFSAPGVRSIL

SEQ ID NO: 27 - protein, 15 aa, Artificial Sequence
  misc_feature: Novel Sequence
HGRFILPWWYAFSPS

SEQ ID NO: 28 - protein, 15 aa, Artificial Sequence
  misc_feature: Novel Sequence
FARYLFTHWWRLPVD

SEQ ID NO: 29 - protein, 15 aa, Artificial Sequence
  misc_feature: Novel Sequence
RYLFYSVHPWRVSYS

SEQ ID NO: 30 - protein, 15 aa, Artificial Sequence
  misc_feature: Novel Sequence
ARVSFWRYSSFAPTY

SEQ ID NO: 31 - protein, 15 aa, Artificial Sequence
  misc_feature: Novel Sequence
IMILLIFSLLWFGGA

SEQ ID NO: 32 - protein, 15 aa, Artificial Sequence
  misc_feature: Novel Sequence
TVGASFWWLSGGKVP

SEQ ID NO: 33 - protein, 15 aa, Artificial Sequence
  misc_feature: Novel Sequence
GRVASMFGGYFFFSR

SEQ ID NO: 34 - protein, 15 aa, Artificial Sequence
  misc_feature: Novel Sequence
WPYLRFSPWVVSPLG

SEQ ID NO: 35 - protein, 15 aa, Artificial Sequence
  misc_feature: Novel Sequence
TSVNRGFLLQRVSHP

SEQ ID NO: 36 - protein, 15 aa, Artificial Sequence
  misc_feature: Novel Sequence
ARFRHSTKSAQFVPL

SEQ ID NO: 37 - protein, 15 aa, Artificial Sequence
  misc_feature: Novel Sequence
GVVWRYTAPVHLGDG

SEQ ID NO: 38 - protein, 15 aa, Artificial Sequence
  misc_feature: Novel Sequence
LDVVLAWRDGLSGAS

SEQ ID NO: 39 - protein, 20 aa, Artificial Sequence
  misc_feature: Novel Sequence
GGIYYPYDIYYPYDIYYPYD

SEQ ID NO: 40 - protein, 20 aa, Artificial Sequence
  misc_feature: Novel Sequence
GGIYWRYDIYWRYDIYWRYD

SEQ ID NO: 41 - protein, 20 aa, Artificial Sequence
  misc_feature: Novel Sequence
GGIYYRYDIYYRYDIYYRYD

SEQ ID NO: 42 - protein, 14 aa, Artificial Sequence
  misc_feature: Novel Sequence
GSSFWRYTTYYDPS

SEQ ID NO: 43 - protein, 15 aa, Artificial Sequence
  misc_feature: Novel Sequence
IMILLIFSLLWFGGA

SEQ ID NO: 44 - protein, 15 aa, Artificial Sequence
  misc_feature: Novel Sequence
GDTRYIPALQHGDKK

SEQ ID NO: 45 - protein, 15 aa, Artificial Sequence
  misc_feature: Novel Sequence
CKGNFGCSSLVFIRA

SEQ ID NO: 46 - protein, 15 aa, Artificial Sequence
  misc_feature: Novel Sequence
GSGGLVLSRISVLLG

SEQ ID NO: 47 - protein, 15 aa, Artificial Sequence
  misc_feature: Novel Sequence
WPHVSGGPGLPMVFF

SEQ ID NO: 48 - protein, 15 aa, Artificial Sequence
  misc_feature: Novel Sequence
YGVPLVNFPNAVVRG

SEQ ID NO: 49 - protein, 15 aa, Artificial Sequence
  misc_feature: Novel Sequence
VPWAGSSLYAILVPT

SEQ ID NO: 50 - protein, 15 aa, Artificial Sequence
  misc_feature: Novel Sequence
AASAHALFSPPFALR

SEQ ID NO: 51 - protein, 15 aa, Artificial Sequence
  misc_feature: Novel Sequence
GEASGLCCRWSLRVV

SEQ ID NO: 52 - protein, 15 aa, Artificial Sequence
  misc_feature: Novel Sequence
VFQFRASVGGSHTVI

SEQ ID NO: 53 - protein, 15 aa, Artificial Sequence
  misc_feature: Novel Sequence
LDVVLAWRDGLSGAS

SEQ ID NO: 54 - protein, 15 aa, Artificial Sequence
  misc_feature: Novel Sequence
GVVWRYTAPVHLGDG

SEQ ID NO: 55 - protein, 15 aa, Artificial Sequence
  misc_feature: Novel Sequence
TAWCSLLGCWSFSIG

SEQ ID NO: 56 - protein, 15 aa, Artificial Sequence
  misc_feature: Novel Sequence
ATALLFGSFSQYSNA

SEQ ID NO: 57 - protein, 13 aa, Artificial Sequence
  misc_feature: Novel Sequence
VSIGFYGRVQYHS

SEQ ID NO: 58 - protein, 15 aa, Artificial Sequence
  misc_feature: Novel Sequence
VRVRLYSSHGFQWDV

SEQ ID NO: 59 - protein, 15 aa, Artificial Sequence
  misc_feature: Novel Sequence
TPLPHFSFRHYHPGH

SEQ ID NO: 60 - protein, 15 aa, Artificial Sequence
  misc_feature: Novel Sequence
RNVPPIFNDVYWIAF

SEQ ID NO: 61 - protein, 4 aa, Artificial Sequence
  misc_feature (3)..(3): X is any amino acid
WYXY

SEQ ID NO: 62 - DNA, 60 bp, Artificial Sequence
  misc_feature: Novel Sequence
agcttccaccatgaggtacatgattttaggcttgctcgcccttgcggcagtctgcagcgc

SEQ ID NO: 63 - DNA, 119 bp, Artificial Sequence
  misc_feature: Novel Sequence
ggccgcgaagcagatcatcaacatgtggcaggaggtgggcaaggccatgtacgccggcggcatctactggcgctacgacatctactggcgctacgacatctactggaggtacgactaat

SEQ ID NO: 64 - DNA, 92 bp, Artificial Sequence
  misc_feature: Novel Sequence
ggccgcgaagcagatcatcaacatgtggcaggaggtgggcaaggccatgtacgcccgcatctactaccgctacgacggcttcgcctactaat

SEQ ID NO: 65 - protein, 18 aa, Artificial Sequence
  misc_feature: Novel Sequence
MRYMILGLLALAAVCSAA

SEQ ID NO: 66 - protein, 16 aa, Artificial Sequence
  misc_feature: Novel Sequence
KQIINMWQEVGKAMYA

SEQ ID NO: 67 - protein, 12 aa, Artificial Sequence
  misc_feature: Novel Sequence
ARIYYRYDGFAY

SEQ ID NO: 68 - protein, 16 aa, Artificial Sequence
  misc_feature: Novel Sequence
GGIYWRYDIYWRYDIY

SEQ ID NO: 69 - protein, 16 aa, Artificial Sequence
  misc_feature: Novel Sequence
GGIYWRYDIYWRYDIY

SEQ ID NO: 70 - protein, 18 aa, Artificial Sequence
  misc_feature: Novel Sequence
MRYMILGLLALAAVCSAA

(19) United States
(12) Patent Application Publication
(10) Pub. No.: US 2003/0017497 A1
(43) Pub. Date: Jan. 23, 2003

(54) Peptide mimotopes of carbohydrate antigens

(76) Inventors: Thomas Kieber-Emmons, Newtown Square, PA (US); David B. Weiner, Merion Station, PA (US); Behjatolah Monzavi-Karbassi, Philadelphia, PA (US)

Correspondence Address: Mark DeLuca Esq., Woodcock Washburn Kurtz, Mackiewicz & Norris LLP, One Liberty Place - 46th Floor, Philadelphia, PA, 19103, US

(21) Appl. No.: 09/894,594

(22) Filed: Jun. 28, 2001

Related U.S. Application Data
(63) Continuation-in-part of application No. 09/601,558, filed on Nov. 7, 2000.
Application No. 09/601,558 is a 371 of international application No. PCT/US99/02405, filed on Feb. 4, 1999.
(60) Provisional application No. 60/073,690, filed on Feb. 4, 1998.
(60) Provisional application No. 60/214,517, filed on Jun. 28, 2000.

(51) Int. Cl. G01N 33/53; G01N 33/574; C07K 14/00; C12P 21/04

(52) U.S. Cl. 435/7.1; 435/7.23; 435/70.21; 530/322; 530/324

(57) ABSTRACT

Methods of preparing a peptide and antigenic antibodies which mimic an antigenic carbohydrate are disclosed. Methods of generating an immune response against a pathogen or tumor cell in an individual using such peptides, recombinant antibodies comprising such peptide, or DNA vaccines live attenuated vaccines, or recombinant vaccines that encode such peptides are disclosed. Methods of enhancing binding of anti-antigenic carbohydrate antibodies to the antigenic carbohydrate in an individual and methods of inhibiting binding of a ligand to a receptor which is an antigenic carbohydrate are disclosed. Methods of identifying peptide sequences which can induce an immune response against two or more different pathogens are disclosed. Novel compositions are disclosed.

### FIELD OF THE INVENTION

The present invention relates to compounds that mimic carbohydrate antigens, nucleic acid molecules that encode peptides that mimic carbohydrate antigens, to methods of identifying peptide sequences that mimic carbohydrate antigens, and to uses of compounds that mimic carbohydrate antigens and nucleic acid molecules that encode peptides that mimic carbohydrate antigens.

### BACKGROUND OF THE INVENTION

Carbohydrates play an essential role in cell biology being involved in cell-cell communication, cell proliferation and differentiation (cell growth). Aberrant glycosylation is a basis for uncontrolled cell growth, invasiveness and increased metastatic potential. Carbohydrates that influence the metastatic potential of tumor cells are ubiquitous in nature. Glycoconjugates found on tumor cells mediate the adhesion of pathogens and toxins to host cell. Immunochemical studies of sialylated lipo-oligosaccharides (LOS) of the Gram-negative bacteria Neisseria gonorrhea and Neisseria meningitides indicate that they are antigenically and/or chemically identical to lactoneoseries glycosphingolipids (GSL). The core oligosaccharides of lipopolysaccharides (LPS) of Campylobacter jejuni serotypes exhibit mimicry of gangliosides. The O-chain of a number of Helicobacter pylori strains exhibit mimicry of Lewis X (LeX) and Lewis Y (LeY) blood group related antigens. The use of related antigens as a means to induce immune responses have recently been shown for the ganglioside tumor associated antigen GM2 in which LPS containing “GM2-like” GalNAcbetal-4Gal(2-3NeuAc)-Hex oligosaccharides were prepared from Campylobacter jejuni serotypes. These latter studies showed that the bacterial preparation displayed significantly enhanced immune responses to GM2 over purified GM2 preparations.

Most carbohydrate antigens belong to the category of T cell independent antigens that reflect their inability to stimulate MHC class II dependent T cell help. As a consequence, carbohydrates are not capable of induction of a sufficient anamnestic or secondary immune response. Furthermore, antibodies (Ab) produced in response to carbohydrate antigens usually are not of high affinity compared to those produced by responses to peptide or protein antigens. C. neoformans has been identified as a pathogen to which a polysaccharide-protein conjugate vaccine can target. The vaccine elicits protective antibodies in mice, and the goal is to provide vaccination which will result in the production of effective anti-cryptococcal antibodies to prevent disease in patients at risk. The conjugate vaccine against C. neoformans is intended to elicit protective antibody immunity, even though the role of natural antibody immunity in protection against cryptococcosis is uncertain. Newer vaccines against common pathogens could help limit the spread of drug-resistant microorganisms. Dissemination of penicillin-resistant pneumococci has been associated with infection and carriage by young children among whom the current 23-valent pneumococcal polysaccharide vaccine is ineffective in inducing protective immunity. However, the effectiveness of polysaccharide-protein conjugate vaccine to H. influenzae type b (Hib) suggests that a similar conjugate vaccine to S. pneumoniae, if available, could effectively abort childhood infection with antibiotic-resistant pneumococci and thereby limit the spread of these strains. Opportunities for the development of antibody-based strategies include 1) pathogens for which there is no available antimicrobial therapy (e.g., C. parvum and vancomycin-resistant enterococci); 2) pathogens that affect primarily immunocompromised patients in whom antimicrobial therapy is not very effective (e.g., invasive fungal infections); 3) pathogens for which drug-resistant variants are rapidly spreading (e.g., Pseudomonas aeruginosa; and 4) highly virulent pathogens for which few effective antimicrobial agents are available (e.g., methicillin-resistant S. aureus). Nevertheless, limitations observed for some carbohydrates include: difficulty in antigen purification or synthesis, the utility of carbohydrate carrier-protein coupling strategies that might prove to be impractical for broad application, the possible lack of tumor reactive Abs and the general lack of persistent high titer cytotoxic antibodies in many patients. Consequently, new carbohydrate immunogens, formulations, and alternative vaccination strategies are needed and constantly being evaluated.

Carbohydrate-conjugate vaccines could have application in tumor immunity and cancer therapy because many tumor cells are coated with carbohydrates or glycoproteins. A number of cell surface antigens, including mucins, oncoproteins and carbohydrate antigens have been found to elicit a humoral immune response and, in some instances, circulating immune complexes are observed. An indication that a serological response can be beneficial comes from vaccination studies in melanoma patients in which anti-ganglioside or anti-idiotypic antibodies are associated with a better prognosis in melanoma patients. It is the expectation, based upon evidence from carbohydrate vaccination trials that antibodies can play a role in vivo in tumor regression, potentially opsonizing tumor cells to prevent extravasion, intravasion and metastatic potential.

Historically in the cancer area, emphasis has been placed on developing vaccines capable of inducing higher titer and long-lasting humoral responses against human tumor associated carbohydrate antigens. Carbohydrate formulations that are in the clinic predominantly induce humoral responses that are considered beneficial because they mediate complement dependent cytotoxicity (CDC) or antibody dependent cytotoxicity (ADCC). IgM antibody production after vaccination better correlates with improved survival than IgG production. It is believed that IgM may not be therapeutically beneficial in spite of consistent documentation of the clinical benefit of anti-ganglioside IgM antibodies. This misconception is based on the immune response to protein antigens, which suggest that the IgM response is only transient and not persistent. The binding of an antibody to a cell surface is governed by the density of antigen expressed. This is particularly true for IgM antibodies. The pentameric and hexameric nature of IgM facilitate binding to clusters of antigens. Unbound IgM exists in a planar conformation in solution. IgM changes from planar to staple conformation when it binds to clustered epitopes. The staple conformation facilitates complement fixation and complement-mediated lysis. IgM antibodies induced in patients may not bind to normal cells (lymphocytes, granulocytes or monocytes) because the antigens on the surface of normal cells are expressed in low concentrations or are sparsely distributed. Without sufficient density of epitopes, IgM may not bind to cause the staple conformation.

The role of IgG is understudied because carbohydrates as T cell independent antigens do not induce IgG responses on their own. Extensive T cell help is required. Even with the coupling of carbohydrates with immunological carriers, functional IgG responses are often limited. Therefore, better ways to manipulate both IgG and IgM are needed. In this invention we discuss the utility of peptides as mimotopes of carbohydrates as one alternative.

The production of anti-bacterial vaccines capable of generating long-lasting immunity in all age groups is still a high priority worldwide. A major goal for effective vaccine development is to identify bacterial antigens that can stimulate protective antibodies. The main targets of the protective immune response against bacterial infections are the capsular polysaccharide (PS) as well as the O-Antigen carbohydrate moiety of the lipopolysaccharide (LPS). LPS is usually the major glycolipid present in Gram-negative bacteria. Lipooligosaccharides (LOSs) are the major glycolipids expressed on mucosal Gram-negative bacteria, including members of the genera Neisseria, Haemophilus, Bordatella, and Branhamella. They can also be expressed on some enteric bacteria such as Campylobacter jejuni and Campylobacter coli strains. LOS is analogous to the lipopolysaccharide (LPS) found in other Gram-negative families.

Children under the age of two years do not at all or only poorly respond to an infection with encapsulated bacteria or after vaccination with purified capsular polysaccharide antigen. In contrast, protein antigens, e.g. tetanus- and diphtheriae-toxoid, are good immunogens in this age group. The difference between polysaccharide antigen and protein antigen is that the former are T-dependent antigens whereas the latter are T-independent. The reason for this age dependent “immunodeficiency” is not clear. A functional immaturity of a B-cell population seems to be the reason for the unresponsiveness of young children against TI-2 antigens. Improved immunogenicity to PS is observed when PS is covalently attached to immunogenic carrier proteins (conjugates). TD protein vaccines can become a decisive factor in situations where the responding immune system is immature or suppressed. The Haemophilus conjugate vaccine contains the capsular polysaccharide chemically conjugated to a carrier protein. This results in an immune response against the polysaccharide with characteristics of a T dependent antigen, giving high immunogenicity even in children under the age of two years, resulting in high antibody-production and the induction of immunological memory. However, fundamental differences in both antibody family types, content and antibody quality elicited by different Hib PS conjugate vaccines are observed indicating a fundamental drawback to vaccine development. The effectiveness of polysaccharide-protein conjugate vaccine to H. influenzae type b suggests that a similar conjugate vaccine to S. pneumoniae, if available, could effectively abort childhood infection with antibiotic-resistant pneumococci and thereby limit the spread of these strains. However, Pneumococcal vaccines containing protein-conjugated oligosaccharides may offer no advantage over currently licensed preparations containing unconjugated polysaccharides for immunization of healthy older adults. In addition, a negative potential effect of prior exposure to the carrier protein could suppress antibody response to the polysaccharide administered in a PS-protein conjugate. Subsequently, carriers in which the host has not been sensitized towards might be a more effective formulation.

A large number of encapsulated bacteria cause human disease and individual vaccines must be developed for each. PS-conjugate vaccines differ chemically and immunologically. These properties translate into fundamental differences in antibody quality elicited by different PS conjugate vaccines. In some cases oligosaccharides that react with or elicit protective antibodies are not available. Consequently, alternative antigens that might function as immunological surrogates are constantly being evaluated. From a vaccine design viewpoint it would be of interest to develop a vaccine that is cost effective. This could be especially important in the developing world where the cost of successful vaccines must be no more than pennies per dose. Formulations that induce cross-reactive immune responses to multiple carbohydrate antigens would consequently be beneficial.

Neisseria and Haemophilus spp. produce a low-M LPS (LOS). LOSs lack O-antigen units with the LOS oligosaccharide structures limited to 10 saccharide units. Other distinguishing characteristics of LOS are the structural and antigenic similarity of some LOS species to human glycolipids and the potential for certain LOSs to be modified in vivo by host substances or secretions. Bacteria with LOS that mimic structures of glycosphingolipids are shown in Table 1. Immunochemical studies of sialylated LOS of the Gram-negative bacteria Neisseria gonorrhea and Neisseria meningitides is antigenically and/or chemically identical to lactoneoseries glycosphingolipids. The terminal trisaccharide lactotriaose (Galb1-4GlcNAcb1-3Gal) is common among LOS of pathogenic Neisseria spp. since both serological and structural studies have shown that most serogroup B and C strains of meningococci and most gonococci strains possess this trisaccharide. Lactotriaose is a precursor of lacto-N-neotetraose, the precursor of lactoneoseries glycosphingolipids. In contrast, the O-chain of a number of Helicobacter pylori strains exhibit mimicry of Lewis X (LeX;Galb1<<4(Fuca1<<3)GlcNAcb1<3Galb1<<4Glcb1<R) and Lewis Y (LeY; Galb1<<4(Fuca1<<3)GlcNAcb1<<3Galb1<<4Glcb1<1R), blood group antigens. Furthermore, N. gonorrhea 1291 has an LOS containing a paragloboside terminal oligosaccharide. Globotriaose (Gala1-4Galb1-4Glc) has been identified as the terminal trisaccharide LOS of the pycocin-resistant mutant. An additional type of mimicry of N. gonorrhoeae involves a pentasaccharide with an N-acetylgalactosamine (GaINAc) residue b 1-3 linked to a terminal galactose (Gal) of a lacto-N-neotetraose. Anti-lactoneoseries monoclonal antibodies bind to Haemophilus ducreyi and to non-typable and type b. H. influenza. The core oligosaccharides of LPS of Campylobacter jejuni serotypes exhibit mimicry of gangliosides.

The blood group-related neolactoseries carbohydrate structures Lewis X (LeX), sialyl-LeX (sLeX), ABH, Lewis a (Lea), sialyl-Lea (sLea) and LeY are examples of terminal carbohydrate structures related to tumor prognosis. These antigens constitute carbohydrate moieties of tumor associated gangliosides, the human carcinoembryonic antigen family, the human pancreatic MUC-1 antigen and are identified in carcinomas of the skin, stomach, pancreas, lung, colon, breast and prostate. The histo-blood group related antigens sLeX and sLea, are also implicated as immunogenic antigens in human melanoma. Melanoma patients immunized with a melanoma cell vaccine (MCV) expressing these antigens developed high titers of IgM but not IgG to both ligands. IgM titers in normal subjects were found to be low. It is noteworthy that patients thus far who developed high titers of anti-sLe antigen IgM showed no evidence of hematologic toxicity (hemolysis, anuria or granulocytopenia, despite the notion that these antigen types are displayed ubiquitously. The core components of Le antigens are structurally very similar among themselves and with LOS and LPS constituents.

### SUMMARY OF THE INVENTION

The present invention relates to methods of preparing a peptide which mimics an antigenic carbohydrate. The method comprises the steps of identifying a peptide sequence which is immunogenically cross reactive with an antigenic carbohydrate and synthesizing a peptide comprising at least two repeating units of the peptide sequence.

The present invention relates to methods of preparing a recombinant antibody which mimics an antigenic carbohydrate the methods comprise the steps of identifying a peptide sequence which is immunogenically cross reactive with an antigenic carbohydrate and synthesizing a recombinant antibody comprising the peptide sequence.

The present invention relates to methods of generating an immune response against a pathogen or tumor cell in an individual. The methods comprise administering to the individual a peptide comprising at least two repeat units of a peptide sequence which is immunogenically cross reactive with an antigenic carbohydrate and/or a recombinant antibody which comprises a peptide sequence which is immunogenically cross reactive with an antigenic carbohydrate and/or a nucleic acid molecule which encodes a peptide sequence which is immunogenically cross reactive with an antigenic carbohydrate.

The present invention relates to methods of enhancing binding of anti-antigenic carbohydrate antibodies to the antigenic carbohydrate in an individual. The methods comprise administering to the individual anti-antigenic carbohydrate antibodies and a peptide comprising one unit or two or more repeat units of a peptide sequence which mimics an antigenic carbohydrate.

The present invention relates to methods of inhibiting binding of a ligand to a receptor which is an antigenic carbohydrate. The methods comprise administering to the individual a peptide comprising one unit or two or more repeat units of a peptide sequence which mimics an antigenic carbohydrate.

The present invention relates to methods of identifying peptide sequences which can induce an immune response against two or more different pathogens. The methods comprise the steps of identifying a peptide sequence which is immunogenically cross reactive with an antigenic carbohydrate which is associated with a pathogen, administering an amount of a peptide comprising the peptide sequence to an animal sufficient to induce an immune response against the peptide, and analyzing the immune response to identify two or more pathogens against which the immune response cross reacts.

The present invention relates to compositions of peptides or proteins containing sequences shown in the Sequence Listing section.

The present invention relates to a nucleic acid molecule that encodes a peptide which mimics an antigenic carbohydrate. The nucleic acid molecule encodes a peptide sequence which is immunogenically cross reactive with an antigenic carbohydrate. In some embodiments, the nucleic acid molecule encodes a peptide which comprises at least two repeating units of the peptide sequence.

The present invention relates to compositions that comprise a nucleic acid molecule that encodes a peptide or protein selected from the group consisting of the peptides or proteins containing sequences shown in the Sequence Listing section.

The basis of this invention is that peptides that mimic carbohydrates can convert otherwise T-independent antigen structures into T-cell dependent antigens, to augment immune responses that target important, yet encryptic carbohydrate targets on pathogenic organisms and on tumor cells. A peptide that mimics a carbohydrate antigen might be formulated to develop a longer lasting immune response. Subsequently peptides that mimic carbohydrates would be of importance as novel agents for vaccine development and in adjuvant therapy. The primary object of the present invention is to produce antigenic peptides which mimic the immunogenic determinants of core structures of GSL, LPS, LOS, hist-blood group related antigens and gangliosides. In accordance with the present invention, monoclonal antibodies specific for neutralizing carbohydrate epitopes are used to identify candidate peptides for use as surrogates for carbohydrate vaccine development. More particularly the present invention discloses antigenic peptides that mimic the immunogenic determinants associated with the envelope protein of human immunodeficiency virus, a variety of bacteria and a variety of human cancers. In a further embodiment of the present invention, there is disclosed a method for inducing a differential immune response eliciting either IgG or IgM polyclonal immune responses. In a further embodiment of the present invention, there is disclosed a method for identifying peptides that mimic carbohydrates using molecular modeling and computational energy calculations.

### BRIEF DESCRIPTION OF THE FIGURES

FIG. 1 depicts the structural similarity of the core components of Le antigens. Fuc: L-frucose; Gal: D-galactose; GlcNAc: N-acetylglucosamine.

FIGS. 2A and 2B depict the reactivity of putative motifs by ELISA. FIG. 2A shows the reactions of MAbs with respective MAP peptide forms. FIG. 2B shows the inhibition of mAb BR55-2 binding to solid-phase LeY-PAA by soluble MAP peptides. Constant amounts of BR55-2 were incubates with increasing amounts of MAP peptides, and then reaction of free mAb with LeY was measured by ELISA. Data points reflect 50% inhibition at 2 μg/ml of peptide inhibitor as measured by reduction of O.D. values in ELISA.

FIG. 3 depicts the reactivity of the anti-sLex antibody FH-6 with MAP peptides.

FIGS. 4A and 4B depict the reaction of anti-peptide sera with LeY and LeB. FIG. 4A shows the reactivity of the IgG portion of peptide-proteosome derived antipeptide sera. FIG. 4B shows the reactivity of the IgM portion derived from MAP immunizations. Pre-immune reactivity of IgM with the carbohydrate probes was subtracted from the respective data points in FIG. 4B.

FIGS. 5A and 5B depict the profile of cross-reactivity of anti-peptide sera with carbohydrate probes. FIG. 5A shows IgG anti-peptide reactivity. FIG. 5B shows carbohydrate-PAA reactive with IgM anti-peptide sear. Pre-immune reactivity of the IgM fraction with the carbohydrate probes was subtracted from the respective data points in FIG. 5B.

FIGS. 6A and 6B depict FACs results for antisera binding to breast and melanoma cells before and after neuraminadase treatment. FIG. 6A shows pre- and post-treatment of SKBR3 cells. FIG. 6B shows pre- and post-treatment of WM793 cells. The P1 sera corresponds to the YYPYD (SEQ ID NO: 6) motif, and P2 corresponds to the YYRYD (SEQ ID NO: 7) motif. Sera in both assays are diluted 1:100.

FIGS. 7A, 7B and 7C depict anti-peptide sera mediation of HIV-1 cell free neutralization. Sera from Balb/c and C57B1/6 mice immunized with P1 or P2, neutralized HIV-1/MN at final dilution's up to 1:64 FIG. 7A shows the neutralization of HIV-1/MN by anti-P1 and P2 sera from Balb/c mice. FIG. 7B shows the neutralization of HIV-1/MN by anti-P1 and P2 sera from C57BI/6 mice. FIG. 7C shows isolate specificity determined by cell free neutralization of the HIV-1/3B isolate.

FIG. 8 depicts the alignment of isolated peptide families reactive with BR55-2 or BR-15-6A

FIG. 9 depicts the specificity on binding among Le antigen probes and constituents by anti-B 1 MAP derived sera. A phage library displaying 15 amino acids to screen the anti-Lewis Y antibodies BR55-2 and BR-15-6A and the anti-GD2/GD3 antibody ME36. 1. The initial choice of using the 15 mer library was predicated on the notion that his length is similar to complimentarity determining regions (CDR) in antibodies which confer mimicry capacity to many anti-idiotypic antibodies.

FIG. 10 depicts the alignment of isolated peptide families reactive with ME361.

FIG. 11 shows a schematic presentation of an expression cassette. Insert is cloned under the control of CMV promoter in pcDNA3 vector. Encoded amino acid sequences for pcDNAggi and pcDNAari are shown. BGH pA indicates BGH polyadenylation site. In amino acid sequence bold shows signal sequence, italic, the additional alanines resulting from inclusion of a restriction site; underlined, mimotopes.

FIGS. 12A and 12B show serum responses to minigen immunization. FIG. 12 A shows pooled sera from four mice per group immunized with pcDNAggi, pcDNAari and pVHSOL was diluted 1:10 and their binding to LeY was studied and compared to naive and vector immunized mice by ELISA. Sera were collected three weeks after the immunization. Bars indicate the standard deviation of averages. Data represent one experiment out of two with comparable results. FIG. 12B shows kinetic of serum reactivity after DNA immunization with designed constructs and vector alone. Sera were collected, pooled, diluted 1:50 and binding to LeY was determined 2, 3 and 4 weeks after immunization. Comparable results were obtained in at least three experiments.

FIG. 13 shows data from groups of mice, four mice in each, that were immunized with LeY or 106 peptide. Mice were bled and collected blood pooled. In a standard ELISA assay reactivity of sera was detected against LeY. The ELISA was done using goat anti mouse IgG or IgM as secondary antibodies.

FIG. 14 shows isotyping kinetic of antibody responses in pcDNAggi immunized mice. Sera of 4 mice were collected and pooled from week 2 to week 7 after immunization. Pooled sera was diluted 1:50 and their binding to LeY was assessed by IgG1 and IgG2a and Igg2b. Standard deviation bars were estimated based on three replications. Experiment repeated twice with similar results.

FIGS. 15A, 15B and 15C show serum reactivity with LeY before and after LeY boost. FIG. 15A) Immunized mice were bled at week eight, boosted with carbohydrate and bled one (week 9) and three weeks (week 11) after boost. Collected sera from pooled blood were diluted 1:50 and studied for binding to LeY by ELISA using goat anti mouse IgM (FIG. 15A) and IgG (FIG. 15B) as secondary antibodies. Anti LeY IgG reactivity of all sera from 11^{th} week was determined (15C). Bars indicate standard deviations and whole experiment repeated twice with expected results.

FIG. 16 shows anti LeY isotypes in boosted mice. Isotype components of collected sera three weeks after carbohydrate boost were determined in 1:50 serum dilution.

FIG. 17 shows communications of pcDNAggi with IL-12 increases T helper activity. Mice were immunized twice bi-weekly and two weeks after the second injection spleens removed. Splenocytes were activated in vitro using 106 MAP for three days.

FIGS. 18A and 18B show IL-12 communications with pcDNAggi induces a significant CTL activity. Mice were immunized as in FIG. 17. Splenocytes were stimulated for 5 days by peptide-pulsed p815 cells. Cytotoxicity was measured on peptide pulsed p815 cells as targets.

FIG. 19 shows serum reactivity data from Example 10.

FIG. 20 shows titration of the anti-LeY immune response data from Example 10.

FIG. 21 shows immune response isotype data from Example 10.

FIG. 22 shows immune response data from Example 10.

FIG. 23 shows immune response data from Example 10.

FIG. 24 shows IgG titer data from Example 10.

FIG. 25 shows immune response isotype data from Example 10.

FIG. 26 shows cross reactive antibody response to glycosylated gp120 data from Example 10.

FIG. 27 shows antibody titer against glycosylated gp120 data from Example 10.

### DETAILED DESCRIPTION OF PREFERRED EMBODIMENTS

Peptide sequences that can antigenically mimic carbohydrates can be identified by those having ordinary skill in the art using peptide-phage display libraries following the previously described methods such as those disclosed in Hoess, R. et al., Gene 128:43-49 (1993), which is incorporated herein by reference or using anti-idiotypic antibodies following the previously described methods such as those disclosed in Westerlink, M. A. J. et al., Proc. Natl. Acad. Sci. USA 92:4021-4025 (1995), which is incorporated herein by reference.

One aspect of the present invention relates to methods of preparing a peptide that mimics an antigenic carbohydrate. The methods comprise the steps of identifying a peptide sequence that immunologically mimics an antigenic carbohydrate and synthesizing a peptide comprising at least two repeating units of the peptide sequence. In some embodiments, the peptide sequence is identified by screening a peptide library with an antibody that is specific for the antigenic carbohydrate. In some embodiments, the antigenic carbohydrate is selected from the group consisting of: histo-blood group related antigens, gangliosides, glycosphingolipids, lipopolysaccharides and lipooligosaccharides. In some embodiments, the antigenic carbohydrate is associated with a pathogen. Examples of antigenic carbohydrates associated with a pathogen include antigenic carbohydrates associated with Gram negative bacteria. Examples of antigenic carbohydrates associated with a pathogen include antigenic carbohydrates associated Neisseria gonorrhea, Neisseria meningitides, Campylobacter jejuni, Helicobacter pylori, C. neoformans, H. influenzae, S. pneumoniae, C. parvum, Pseudomonas aeruginosa, S. aureus, Bordatella spp., Branhamella spp., Campylobacter coli, Haemophilus ducreyi or Cryptococcus neoformans. In some embodiments, the antigenic carbohydrate is associated with a tumor cell. Examples of tumor cells that comprise antigenic carbohydrate include adenocarcinomas and melanomas. The number of repeat units is not critical and it is possible that proteins with multiple copies can be produced. In some embodiments, the peptide comprise three repeating units of the peptide sequence. In some embodiments, the peptide comprise up to 10 repeating units of the peptide sequence. In some embodiments, the peptide comprise more than 10 repeating units of the peptide sequence. In some embodiments, the peptide consists of three repeating units of the peptide sequence. The size of the peptide is not critical and it is possible that it can be a polypeptide or protein. In some embodiments, the peptide consists of up to 28 amino acid residues. In some embodiments, the peptide consists of up to 50 amino acid residues. Peptides may be used according to the invention such as immunogenic agents, vaccine, adjuvants, enhancers of antibody binding, and inhibitors of receptor/ligand binding.

According to some embodiments of the present invention, peptides which mimic an antigenic carbohydrate may be prepared by first identifying a peptide sequence that antigenically mimics with an antigenic carbohydrate. The sequence so identified may be structurally compared to the structure of the antigenic carbohydrate. The sequence may be modified such as, for example, substituting amino acids, insertion of spacer molecules including spacer amino acids, deletion of amino acids, which result in peptide structures more similar to the structure of the antigenic carbohydrate. Peptides which include the modifications are synthesized and such peptides may be used according to the invention such as immunogenic agents, vaccine, adjuvants, enhancers of antibody binding, and inhibitors of receptor/ligand binding. and using that sequence to synthesizing a peptide comprising at least two repeating units of the peptide sequence. The number of repeat units is not critical and it is possible that proteins with multiple copies can be produced. In some embodiments, the peptide comprise three repeating units of the peptide sequence. In some embodiments, the peptide comprise up to 10 repeating units of the peptide sequence. In some embodiments, the peptide consists of three repeating units of the peptide sequence. The size of the peptide is not critical and it is possible that it can be a polypeptide or protein. In some embodiments, the peptide consists of up to 28 amino acid residues. In some embodiments, the peptide consists of up to 50 amino acid residues.

Another aspect of the present invention relates to methods of preparing a recombinant antibody which mimics an antigenic carbohydrate, and to recombinant antibodies which mimic antigenic carbohydrates. The methods comprise the steps of identifying a peptide sequence that induces an immunogenically cross reactive response with an antigenic carbohydrate and synthesizing a recombinant antibody comprising the peptide sequence. Peptides comprising the peptide sequence induce an immune response that cross-reacts with a carbohydrate antigen. In some embodiments, the peptide sequence is identified by screening a peptide library with an antibody against the antigenic carbohydrate. In some embodiments, the antigenic carbohydrate is selected from the group consisting of: histo-blood group related antigens, gangliosides, glycosphingolipids, lipopolysaccharides and lipooligosaccharides. In some embodiments, the antigenic carbohydrate is associated with a pathogen. In some embodiments, the antigenic carbohydrate is associated with a pathogen selected from the group consisting of: Neisseria gonorrhea, Neisseria meningitides, Campylobacter jejuni, Helicobacter pylori, C. neoformans, H. influenzae, S. pneumoniae, C. parvum, Pseudomonas aeruginosa, S. aureus, Bordatella spp., Branhamella spp., Campylobacter coli, Haemophilus ducreyi or Cryptococcus neoformans. In some embodiments, the antigenic carbohydrate is associated with a tumor cell. In some embodiments, the recombinant antibody comprises a variable region that comprises the peptide sequence. In some embodiments, the recombinant antibody comprises one or more complementarity determining regions that comprises the peptide sequence. In some embodiments, the recombinant antibody comprises human antibody sequences. In some embodiments, the recombinant antibody comprises a human antibody with the peptide sequence that mimics the antigenic carbohydrate in the variable region. According to some embodiments of the present invention, the recombinant antibodies include sequences which are two or three repeat units of the peptide sequence identified as immunogenically cross reactive an antigenic carbohydrate. According to some embodiments of the present invention, the recombinant antibodies include sequences which are one to three and up to ten or more repeat units of a peptide sequence which is modified from the peptide sequence identified as immunogenically cross reactive an antigenic carbohydrate. The modifications are designed based upon a structural comparison of the structure of the antigenic carbohydrate with the initial peptide sequence identified as cross reactive. The sequence may be modified such as, for example, substituting amino acids, insertion of spacer molecules including spacer amino acids, deletion of amino acids, which result in peptide structures more similar to the structure of the antigenic carbohydrate.

Another aspect of the present invention relates to methods of generating an immune response against a pathogen or tumor cell in an individual. The methods of the present invention provide for the induction of a differential immune response eliciting either IgG or IgM polyclonal immune responses. The methods comprise administering to an individual peptides described above which comprising at least two repeat units of a peptide sequence which is immunogenically cross reactive an antigenic carbohydrate or a modification thereof and/or a recombinant antibody which comprises a peptide sequence which is immunogenically cross reactive an antigenic carbohydrate or a modification thereof. The peptide sequence may identified by screening a peptide library with an antibody against the antigenic carbohydrate. In some embodiments, the antigenic carbohydrate is selected from the group consisting of: histo-blood group related antigens, gangliosides, lipopolysaccharides and lipooligosaccharides. In some embodiments, the antigenic carbohydrate is associated with a pathogen selected from the group consisting of: Neisseria gonorrhea, Neisseria meningitides, Campylobacter jejuni, Helicobacter pylori, C. neoformans, H. influenzae, S. pneumoniae, C. parvum, Pseudomonas aeruginosa, S. aureus, Bordatella spp., Branhamella spp., Campylobacter coli, Haemophilus ducreyi or Cryptococcus neoformans. In some embodiments, the immune response is generated against a pathogen, the individual is free of infection by the pathogen and the immune response is prophylactic. In some embodiments, the immune response is generated against a pathogen, the individual is infected by the pathogen and the immune response is therapeutic. In some embodiments, the immune response is generated against a tumor cell, the individual is free of the tumor cell and the immune response is prophylactic. In some embodiments, the immune response is generated against a tumor cell, wherein the individual has a tumor comprising the tumor cell and the immune response is therapeutic. The number of repeat units is not critical and it is possible that proteins with multiple copies can be produced. In some embodiments, the peptide comprise three repeating units of the peptide sequence. In some embodiments, the peptide comprise up to 10 repeating units of the peptide sequence. In some embodiments, a peptide that consists of three repeating units of the peptide sequence is administered to the individual. The size of the peptide is not critical and it is possible that it can be a polypeptide or protein. In some embodiments, the peptide consists of up to 28 amino acid residues. In some embodiments, the peptide consists of up to 50 amino acid residues.

Another aspect of the present invention relates to methods of generating an immune response against a pathogen or tumor cell in an individual using DNA vaccines, live, attenuated vaccines or recombinant vaccines. The methods comprise administering to an individual DNA vaccines, live, attenuated vaccines or recombinant vaccines which comprise nucleic acid sequences that encode the peptides described above operably linked to regulatory sequences. Expression of the peptide sequences produces a peptide which is immunogenically cross reactive an antigenic carbohydrate. The peptide sequence may identified by screening a peptide library with an antibody against the antigenic carbohydrate. In some embodiments, the antigenic carbohydrate is selected from the group consisting of: histo-blood group related antigens, gangliosides, lipopolysaccharides and lipooligosaccharides. In some embodiments, the antigenic carbohydrate is associated with a pathogen selected from the group consisting of: Neisseria gonorrhea, Neisseria meningitides, Campylobacter jejuni, Helicobacter pylori, C. neoformans, H. influenzae, S. pneumoniae, C. parvum, Pseudomonas aeruginosa, S. aureus, Bordatella spp., Branhamella spp., Campylobacter coli, Haemophilus ducreyi or Cryptococcus neoformans. In some embodiments, the immune response is generated against a pathogen, the individual is free of infection by the pathogen and the immune response is prophylactic. In some embodiments, the immune response is generated against a pathogen, the individual is infected by the pathogen and the immune response is therapeutic. In some embodiments, the immune response is generated against a tumor cell, the individual is free of the tumor cell and the immune response is prophylactic. In some embodiments, the immune response is generated against a tumor cell, wherein the individual has a tumor comprising the tumor cell and the immune response is therapeutic. The number of repeat units is not critical and it is possible that proteins with multiple copies can be produced. In some embodiments, a peptide that comprises three repeating units of the peptide sequence is administered to the individual. In some embodiments, a peptide that comprises ten or more repeating units of the peptide sequence is administered to the individual. In some embodiments, a peptide that consists of one, two or three repeating units of the peptide sequence is administered to the individual. The size of the peptide is not critical and it is possible that the nucleic acid molecule can encode a peptide that can be a polypeptide or protein. In some embodiments, the peptide consists of up to 28 amino acid residues. In some embodiments, the peptide consists of up to 50 amino acid residues.

One aspect of the invention is using peptides as vaccine adjuvants. The peptides may have one, two or three and up to 10 or more repeat units of a peptide sequence identified as immunogenically cross reactive an antigenic carbohydrate The peptides may be modified to more closely resemble the structure of the antigenic carbohydrate. In some embodiments, the antigenic carbohydrate is associated with a pathogen and the peptide is administered in conjunction with vaccine against the pathogen. In some embodiments, the antigenic carbohydrate is associated with a tumor cell and the peptide is administered in conjunction with vaccine against the tumor cell. In some embodiments, the peptide consists of up to 28 amino acid residues. In some embodiments, the peptide consists of up to 50 amino acid residues. In some embodiments, a recombinant antibody that comprises a variable region that comprises the peptide sequence is administered to the individual. In some embodiments, the recombinant antibody comprises one or more complementarity determining regions that comprises the peptide sequence.

Another aspect of the present invention relates to methods of enhancing binding of anti-antigenic carbohydrate antibodies to the antigenic carbohydrate in an individual. The methods comprise administering to the individual an anti-antigenic carbohydrate antibodies and a peptide comprising one peptide sequence or two or more repeat units of a peptide sequence which mimics an antigenic carbohydrate. The enhancement aspect of the invention may be used in connection with any passive immunotherapy using antibodies directed at an antigenic carbohydrate. In some embodiments, the peptide consists of two or three repeating units of the peptide sequence. In some embodiments, the anti-antigenic carbohydrate antibodies bind to an antigenic carbohydrate on a tumor cell. In some embodiments, the antibody is an anti-Lewis Y antibody, an anti-ganglioside antibody or an antibody specific for an antigenic carbohydrate present on a virus such as HIV.

One of skill in the art of antibody production can prepare a variety of appropriate antibodies for the present invention with no more than routine experimentation. A number of texts in the field, e,g., Antibody Engineering: A Practical Guide, Carl. A. Borrebaeck, ed., W.H. Freeman and Co., New York and Antibodies: A Laboratory Manual, Ed Harlow and David Lane, eds., Cold Spring Harbor Laboratory, New York, latest editions, provide considerable guidance in this respect.

Another aspect of the present invention relates to methods of inhibiting binding of a ligand to a receptor which is an antigenic carbohydrate. The methods comprise administering to the individual a peptide comprising 1-3 or more repeat units of a peptide sequence which mimics an antigenic carbohydrate. In some embodiments, the peptide consists of two or three repeating units of the peptide sequence.

A further aspect of the present invention relates to methods of identifying peptide sequences which can induce an immune response against two or more different pathogens. The methods comprise the steps of identifying a peptide sequence which is immunogenically cross reactive an antigenic carbohydrate which is associated with a pathogen, administering an amount of a peptide comprising the peptide sequence to an animal sufficient to induce an immune response against the peptide, and analyzing the immune response to identify two or more pathogens against which the immune response cross reacts. The peptides may comprise one, two, three or more repeat units of a peptide sequence which is immunogenically cross reactive an antigenic carbohydrate or a modification thereof. Recombinant antibody which comprises a peptide sequence which is immunogenically cross reactive an antigenic carbohydrate or a modification thereof may be prepared. The peptide sequence may identified by screening a peptide library with an antibody against the antigenic carbohydrate. In some embodiments, the antigenic carbohydrate is selected from the group consisting of: histo-blood group related antigens, gangliosides, lipopolysaccharides and lipooligosaccharides. In some embodiments, the antigenic carbohydrate is associated with a pathogen selected from the group consisting of: Neisseria gonorrhea, Neisseria meningitides, Campylobacter jejuni, Helicobacter pylori, C. neoformans, H. influenzae, S. pneumoniae, C. parvum, Pseudomonas aeruginosa, S. aureus, Bordatella spp., Branhamella spp., Campylobacter coli, Haemophilus ducreyi or Cryptococcus neoformans. In some embodiments, a peptide that comprises three repeating units of the peptide sequence is administered to the individual. In some embodiments, a peptide that consists of three to ten or more repeating units of the peptide sequence is administered to the individual. In some embodiments, a peptide that consists of three repeating units of the peptide sequence is administered to the individual. In some embodiments the peptide is greater than 50 amino acid residues. In some embodiments, the peptide consists of up to 28 amino acid residues. In some embodiments, the peptide consists of up to 50 amino acid residues. The peptides are analyzed to determine and identify whether the immune response generated against them cross reacts to two or more pathogens. In some embodiments, an animal, such as for example a mouse, rat, rabbit, guinea pig, or primate, is administered the peptide and sera in the animal is analyzed to identify antibodies which are reactive against a panel of pathogen samples.

An aspect of the invention relates to peptides that comprise the sequences of SEQ ID NOS: 1-44 contained within the Sequence Listing section.

The present invention further relates to pharmaceutical composition comprising peptides and a physiologically acceptable carrier.

Peptides can be synthesized by those having ordinary skill in the art using well known techniques and readily available starting materials. Peptides may be produced using any method known in the art, including, but not limited to, chemical synthesis as well as biological synthesis in an in vitro or in vivo in a eukaryotic or prokaryotic expression system. For example, peptides of the invention may be produced by solid phase synthesis techniques as taught by Merryfield, (1963) J. Am. Chem. Soc., 15:2149-2154 and J. Stuart and J. D. Young, Solid Phase Peptide Synthelia, Pierce Chemical Company, Rockford, Ill. (1984), each of which is incorporated herein by reference.

The peptides and recombinant antibodies of the present invention may be administered by oral, intraperitoneal, intramuscular and other conventional routes of pharmaceutical administration. Pharmaceutical compositions of the present invention may be administered either as individual therapeutic/prophylactic agents or in combination with other agents. They can be administered alone, but are generally administered with a pharmaceutical carrier selected on the basis of the chosen route of administration and standard pharmaceutical practice.

The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.0001 to 1 grams per kilogram of body weight, in some embodiments about 0.1 to 100 milligrams per kilogram of body weight. Ordinarily dosages are in the range of 0.5 to 50 milligrams per kilogram of body weight, and preferably 1 to 10 milligrams per kilogram per day. In some embodiments, the pharmaceutical compositions are given in divided doses 1 to 6 times a day or in sustained release form is effective to obtain desired results.

Dosage forms (composition) suitable for internal administration generally contain from about 1 milligram to about 500 milligrams of active ingredient per unit. In these pharmaceutical compositions the active ingredient will ordinarily be present in an amount of about 0.5-95 by weight based on the total weight of the composition.

For parenteral administration, the compound can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques.

Suitable pharmaceutical carriers are described in the most recent edition of Remington's Pharmaceutical Sciences, 18th Edition, A. R. Gennaro et al. Eds., Mack Publishing Co., Easton, Pa., 1990, a standard reference text in this field.

For example, a parenteral composition suitable for administration by injection is prepared by dissolving 1.5% by weight of active ingredient in 0.9% sodium chloride solution.

According to the present invention, the compound may be administered to tissue of an individual by topically or by lavage. The compounds may be formulated as a cream, ointment, salve, douche, suppository or solution for topical administration or irrigation. Formulations for such routes administration of pharmaceutical compositions are well known.

Generally, additives for isotonicity can include sodium chloride, dextrose, mannitol, sorbitol and lactose. In some cases, isotonic solutions such as phosphate buffered saline are used. Stabilizers include gelatin and albumin. In some embodiments, a vasoconstriction agent is added to the formulation. The pharmaceutical preparations according to the present invention are preferably provided sterile and pyrogen free.

One of skill in the art of pharmaceutical formulations, e.g., having an advanced degree in Pharmaceutics or Pharmaceutical Sciences, can prepare a variety of appropriate dosage forms and formulations for the compositions of the invention with no more than routine experimentation. A number of texts in the field, e.g., Remington's Pharmaceutical Sciences and The U.S. Pharmacopoeia/National Formulary, latest editions, provide considerable guidance in this respect.

A pharmaceutically acceptable formulation will provide the active ingredient(s) in proper physical form together with such excipients, diluents, stabilizers, preservatives and other ingredients as are appropriate to the nature and composition of the dosage form and the properties of the drug ingredient(s) in the formulation environment and drug delivery system.

Subsequent to initial administration, individuals may be boosted by readministration. In some preferred embodiments, multiple administrations are performed.

In some embodiments, the peptides are delivered to cells in the form of DNA vaccines. Methods for delivering peptides to cells by direct DNA administration have been reported using a variety of protocols. Examples of such methods are described in U.S. Pat. No. 5,593,972, U.S. Pat. No. 5,739,118, U.S. Pat. No. 5,580,859, U.S. Pat. No. 5,589,466, U.S. Pat. No. 5,703,055, U.S. Pat. No. 5,622,712, U.S. Pat. No. 5,459,127, U.S. Pat. No. 5,676,954, U.S. Pat. No. 5,614,503, and PCT Application PCT/US95/12502, which are each incorporated herein by reference. Briefly, a nucleic acid sequence that encodes the peptide sequence of the peptide to be delivered is inserted into an expression vector which is administered to cells of an individual to be vaccinated. The cells take up the expression vector and produce the peptide. An immune response is generated against the peptide that is expressed.

It is also contemplated that the peptide sequences which mimic the antigenic carbohydrate can be incorporated into attenuated live vaccines and vaccines which use recombinant vectors to deliver foreign genes that encode antigens. Examples of attenuated live vaccines and those using recombinant vectors to deliver foreign antigens are described in U.S. Pat. Nos.: 4,722,848; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; and 5,482,713, which are each incorporated herein by reference. Gene constructs are provided which include the nucleotide sequence that encodes the peptide sequences which mimic the antigenic carbohydrate operably linked to regulatory sequences that can function in the vaccinee to effect expression. The gene constructs are incorporated in the attenuated live vaccines and recombinant vaccines to produce improved vaccines according to the invention.

Lactoseries structures

Three important criteria suggest that lactoseries structures are potential targets for immunotherapy in humans: 1) their specific up-regulation (density of expression) on tumor cells; 2) their function as differentiation antigens; and 3) their role in cell adhesion and motility underlying their metastatic potential. The expression of LeX, LeY and sLeX in neutrophils is limited to humans. Therefore, immune responses against such carbohydrates may be weaker in humans compared to other mammals. LeY, for example, is immunogenic in mice. Tumor associated carbohydrate antigens (including LeY) are expressed at low levels on normal tissues. LeY structures have not been chemically isolated from neutrophils but it is possible neutrophils do express low levels of LeY. The expression of extended LeY with internally fucosylated structure (LeY-LeX) is limited in normal cells and tissues.

While a LeY-conjugate vaccine is attractive for development, antibodies generated against synthetic LeY are not always cross-reactive with native LeY antigen forms. This general phenomenon has also been observed with sTn formulations, suggesting that neoglycoproteins containing sTn in which the carbohydrate structures are clustered together would make better immunogens. Likewise, it has also been observed with GM2-KLH formulations in which the majority of IgG antibodies induced by the GM2-KLH/QS-21 vaccine, while reactive in ELISA, failed to react with cell surface expressed GM2. Synthetic LeY formulations induce anti-synthetic LeY reactivities but the generated antibodies may not bind to tumor cells or bind very weakly. This observation formulates one of the rationales for the proposed studies. Serological analyses have indicated that MAbs made against synthetic LeY (i) react strongly with synthetic LeY but poorly with natural LeY, (ii) cross-react with LeX or H-type 2 structures, and (iii) were IgG1, IgG2a, or IgG2b. MAbs made against LeY-expressing cells (i) react with both synthetic LeY and natural LeY, (ii) were of two types: cross-reactive with LeX or H-type 2 structures or specific for LeY, and (iii) were IgM or IgG3. These results indicate that better ways to synthesize LeY (and related) immunogens that are reflective of naturally expressed LeY (and related) structures or alternative ways to induce immune responses cross-reactive with native LeY (and related structures) are needed.

In an attempt to overcome the problems that arise from the T-independent immune response induced by such antigens, vaccine strategies have focused on development of either polysaccharide-protein conjugates or on anti-idiotypic antibodies that mimic PS. The difficult steps in the former approach are the purification of the polysaccharide (especially when starting from LPS, which must be devoid of any residual lipid A-related endotoxin activity) and the loss of immunogenicity of the carbohydrate moiety during coupling to the protein carrier. Carbohydrate synthesis may diminish the problems associated with antigen purification, but remains a limited solution due to the overall difficulties of carbohydrate chemistry. The latter strategy, based upon the mimicry of carbohydrate antigens by anti-idiotope antibodies, is not a simpler alternative, since obtaining these antibodies is relatively time-consuming and elucidating which ones of many possible anti-idiotypes is also not straight forward.

An alternate approach to the development of T-dependent vaccines for carbohydrates is through the use of peptides that mimic capsular polysaccharides or LPS/LOS. Carbohydrate mimicking peptides could revolutionize vaccines against infectious pathogens. Approaches to identify peptides that mimic carbohydrates have been described. Particularly appealing are those associated with screening peptide phage display libraries with anti-carbohydrate antibodies. Peptides that mimic carbohydrate structure have significant advantages as vaccines compared with carbohydrate-protein conjugates or anti-idiotypic antibodies. First, the chemical composition and purity of synthesized peptides can be precisely defined. Second, the immunogenicity of the peptides can be significantly enhanced by polymerization or addition of relatively small carrier molecules that reduce the total amount of antigen required for immunization. Third, peptide synthesis may be more practical than synthesis of carbohydrate-protein conjugates or the production anti-idiotypes. Fourth peptide mimicking sequences can be engineered into DNA plasmids for DNA vaccination to further manipulate T cell responses.

Peptide mimotopes

Peptide mimotopes of carbohydrate antigens are viable tools to investigate the fine specificity of anti-carbohydrate antibodies. The screening of peptide phage display libraries with anti-carbohydrate antibodies shows that peptide mimotopes for complex microbial polysaccharides can be identified, demonstrating the usefulness of such peptides in analyzing closely related interactive sites of proteins in general and of antibodies in particular. Peptide mimotopes of carbohydrate antigens that can be rendered immunogenic can provide an alternative immunogen for carbohydrate antigens that are difficult to isolate or synthesize. In addition, peptide mimotopes provide an alternative to identifying epitopes that are otherwise not defined chemically as those associated with some complex carbohydrate determinants.

Peptide libraries provide an almost infinite source of molecular shapes, amongst which one would expect to find mimics of any given antigen. Screening of random peptide libraries with monoclonal libraries has selected specific peptides. Such peptides will reflect the conformation of the antigen binding site and may provide molecular mimotopes of particular epitopes. Although peptide libraries have been used to identify mimotopes for a few saccharides, it was not certain that peptide mimotopes could be identified that would bind well enough to inhibit the binding of antibodies to carbohydrate antigens or induce immune responses that are protective in nature.

Peptide mimotopes for carbohydrates have been defined containing a two aromatic amino acid repeat motif W/YXY found to Con A (YPY), in peptides that mimic the Lewis Y antigen (WLY), in peptides that bind to antibodies to the meningococcal group C capsular polysaccharide (YRY), and in antibodies that bind to Crytococcous epitopes. These observations argue that a particular peptide structure is required for polysaccharide mimicry. Antibody heavy chain complementarity regions constitute a natural constrained loop peptide library that are rich in aromatic amino acids, especially tyrosine. Binding site specific anti-anti-idiotypic antibodies can serve as mimotopes for polysaccharide antigens. In this context , the binding site of an anti-idiotypic antibody could be looked upon as a way of presenting peptides so that they will mimic a particular conformation of a non-protein antigen. A more precise understanding of the binding of peptides and saccharides at the molecular level is required in order to determine whether the occurrence of motifs like W/YXY in mimotopes of saccharide structures is due to molecular mimicry or simply reflects an advantage provided by aromatic rings for interactions between proteins. In addition to the role that peptide mimotopes can play in exploring the fine specificity of antibodies, they may mimic polysaccharides as antigen and potentially elicit an anti-oligosaccharide response. Not all peptides that have been isolated from peptide libraries induce an anti-polysaccharide response. The problem now is to identify the modifications of such peptides that are most likely to induce a high anti-polysaccharide response.

A Molecular Basis for Antigenic and Immunogenic Mimicry

With the advent of monoclonal antibodies, identification of similar structures recognized by a single antibody species became greatly simplified. Over the past decade, pioneering work from several laboratories has clearly demonstrated that antigens of different chemical chemicals structures, such as carbohydrates and peptides or nucleic acids and peptides, can efficiently bind to a single antibody molecular species. These findings have profoundly changed the definition of molecular mimicry to include not only amino acid identities or similarities between protein molecules, but also similarities between chemically different molecules. They have also changed the philosophy of immunologists about the way an antibody molecule functions as a receptor to recognize the antigen. It is now evident that one antibody does not necessarily bind to a single antigen, but it may recognize antigens with similarities in structure even though the chemical composition of the antigens may be very different, such as carbohydrates and peptide structures. Systematic approaches improving computational as well as experimental tools have been used to analyze and exploit topological similarity between dissimilar molecules. It is apparent that the structural rules governing molecular mimicry are required to be defined for its successful exploitation. The chemical nature of the mimicry between carbohydrates and mimicking peptide is not completely understood; however recent evidence has shown that aromatic-aromatic and hydrophobic interactions are critical chemical forces between carbohydrate mimicking peptides and their antibody combing site.

An appealing approach to identify potential carbohydrate surrogate antigens is the screening of peptide libraries with carbohydrate reactive antibodies (Ab). Screening against peptide display libraries identifies different molecular species than the one the antibody was raised against. Analysis of a larger repertoire of ligands reactive with an Ab combining site might establish structure/function relationships not evident from monoreactive molecules like anti-Ids. Peptide displayed libraries containing millions of different small peptides are a novel and rich potential source of ligands which might bind specifically to antibodies. An important criteria for selecting antibodies for peptide library screening is that they display a biological activity. Consequently, selection is less biased by affinity considerations since even moderate affinity antibodies are adequate for most studies. Previous work with anti-idiotypic Abs indicates that competition with a target carbohydrate antigen and high affinity binding are often not sufficient for immunological mimicry, and at times, may not be necessary. Only anti-Ids that compete with carbohydrate antigens for binding to the primary Ab (Ab 1), a non-competitive anti-Id, induced the greatest enhancement of carbohydrate reactivity when used along with the carbohydrate for immunization. It was observed that all the anti-Ids elicited the same Ab1 idiotype. Subsequently, it is arguable that the anti-Ids in that study have more to do with eliciting the correct idiotype than with mimicry of carbohydrate. By extension, an embodiment of the invention is that antibodies reactive with native forms of carbohydrate antigens associated with bacteria, fungi, viruses or human tumors can be used to isolate peptides that might mimic the conformational features of the surface expressed antigen that would then be used to immunize animals and humans to generate an immune response reactive with the naturally expressed epitope on the pathogenic organisms or on tumor cells.

There is accumulating evidence that cellular rather than antibody responses are more effective for tumor rejection. The most successful type of vaccine for tumor antigens would elicit not only Th2-type CD4+ T helper cells but also Th1 CD4+ delayed-type hypersensitivity (DTH) T cells and cytotoxic T cells of either CD4+ or CD8+ phenotype. Peptides mimotopes of carbohydrates linked to MHC Class II anchor peptides may manipulate Th1 or Th2 responses. DTH responses have been noted against sialyl-Lewis (sLe) antigens. One suggestion for T cell mediated immunity is through the formation of immune complexes of idiotypes (Id) and anti-ids on tumor surfaces that appear to be targets of T cells augmenting ADCC. Such an explanation was suggested as a mechanism for targeting sialylated LeX antigen expressing tumors. Antibodies directed toward carbohydrates on T cells may also be potent T cell activators. Most studies on human tumor T cell immunity focus on the CD3+CD8+ cytotoxic T lymphocyte (CTL) phenotype; however, CD3+CD4+ CTL are important effector cells in other diseases and may also contribute to anti-tumor immunity. CD4+ T cells are associated with antigen memory response and helper function; therefore activation of CD4+ CTL may be more beneficial regarding long-term protective anti-tumor immunity. Peptide mimotopes of carbohydrates attached to potent immunological carriers may be further manipulated to induce such responses.

Peptides that mimic carbohydrate structure have further significant advantages as vaccines compared with carbohydrate-protein conjugates or anti-idiotypic antibodies. First, the chemical composition and purity of synthesized peptides can be precisely defined. Second, the immunogenicity of the peptides can be significantly enhanced by polymerization or addition of relatively small carrier molecules that reduce the total amount of antigen required for immunization. Third, peptide synthesis may be more practical than synthesis of carbohydrate-protein conjugates or the production of anti-idiotypes. Fourth, peptide mimicking sequences can be engineered into DNA plasmids for DNA vaccination to further manipulate T cell responses.

The present invention discloses a method for identifying candidate peptides for use as surrogate carbohydrate antigens in vaccine development and the composition of said peptides. For identifying both linear and constrained peptide mimotopes, a random peptide bacteriophage or other similar peptide expressing system is first constructed. The peptide library is constructed using conventional procedures in the art. Examples of construction of such libraries include but not limited to those described (Cwirla et al., Proc. Natl. Acad. Sci. USA, 1990, 87: 6378-6382; Scott et al., Science, 1990, 249: 386-390; Scott et al., Trends in Biochemical Sciences, 1992, 17: 241-5) which disclosures are hereby incorporated by reference. Once the library is constructed, it is screened with a monoclonal antibody that exhibits a desired biological specificity like neutralization. Defining the configuration of carbohydrate epitopes as recognized by antibodies is important for understanding the basis for specificity of MAbs, for the development of more effective MAbs, and for designing vaccines against carbohydrates. The basis of the present invention is that antibodies that are specific for carbohydrate antigens and display an important biological finction can be used to identify peptides that mimic important conformational features of carbohydrates that is required to induce effective anti-carbohydrate immune responses. After screening bacteriophage plaques which bind to the antibody are selected, amplified, cloned using standard techniques and large quantities of the bacteriophage are grown and their ability to inhibit binding of the isolating antibody to the nominal antigen is tested. Phage that inhibit binding of the antibody are sequenced to determine the oligonucleotide and amino acid sequences of the cloned peptide. These peptides are then synthesized and used as immunogens to determine whether the peptides are capable of generating antibodies against the nominal antigen and can elicit a protective response against the antigen either in vitro or in animal models.

One strategy for obtaining putative motifs/peptides that are singularly reactive with an antibody is to perform subtractive screening of the libraries. In this approach, phage reactive with one antibody are amplified and allowed to react with other carbohydrate antibodies and irrelevant antibodies with differing carbohydrate specificity [symbol] s. Only those phage reactive with the query antibody are kept for further analysis and sequencing. Selected phage are grown and rescreened against another anti-carbohydrate antibodies with the same or related carbohydrate specificity. Clones are isolated from the two or three pools of enriched phage displaying the highest enrichment and/or ELISA signal for antibody binding, as well as ability to compete with antigen for antibody binding to the carbohydrate antigen. Of these clones, a consensus sequence is looked for. If one is observed the peptide is synthesized and determined whether it displays antibody binding activity and whether it inhibits nominal antigen binding to the query antibody.

Alternatively, if multiple consensus peptides are observed from the phage screen, molecular modeling can be used to identify topological equivalencies to identify the best peptides that match carbohydrate conformations. As above, relative enrichment of phage and/or ELISA signal for antibody binding, as well as ability to compete with antigen for antibody binding to the carbohydrate epitope are the major criteria for selection of peptides for further analysis. The degree of antigenic mimicry is further assessed by using computer modeling to evaluate contact residues and optimal binding conformations between identified putative motifs from the phage screening and residues within the antibody combining site. Comparisons of motifs experimentally observed from phage screening and from the molecular modeling studies, provides information as to differences in the nature of binding by various peptide analogs. Comparing random peptides of isolated phage indicates common essential residues necessary for antibody recognition. Aligning the inserts based on detected homology can reveal consensus sequences that correspond to secondary protein structures that can be correlated with how the putative motif fits within the antibody combining site. Assessment of mimicry is established from (i) the degree of sequence homology with a set of peptides, (ii) the conformational properties of the peptide motifs within the combining site are evaluated by energetic considerations (strength of interaction, number of hydrogen bonds formed, low energy conformational properties in the presence and absence of restraints imposed by the antibody combining site), and (iii) topological relationships between the peptide motif with carbohydrate structures. Topological equivalencies are those in which functional groups on the carbohydrate and motif contact the same residues in the antibody combining site, display similar shape and volume relationships and/or charge relationships. Such equivalencies are typical in pharmacophore development.

Two strategies are taken in the modeling. The first is to explore potential orientations of putative motifs within the antibody combining site using several computer programs and their supplied parameters. The criterion for selection is based upon energetic and geometrical relationships; i.e. intermolecular interaction energy, and stability of the binding mode conformation(s) within the combining site and entropy calculations have been performed for comparison with experimental thermodynamics data). The results of the stimulation can help give an explanation of anticipated affinity constants obtained from affinity measurements by BlAcore analysis (for example) at the molecular level. The steps in this first phase of modeling are: 1) LUDI is implemented to find reactive positions and potential starting configurations as described in our manuscript. 2) The residues in these defined positions are “stitched” together by linking the residues through introduction of backbone atoms between non overlapping residues. Generation of starting models will employ the computer program INSIGHTII (Biosym Technologies). Peptides are computer generated and the backbone is conformationally adjusted so as the residues have maximum overlap with the LUDI positioned fragments. 3) The placement of the peptide is further explored using the program AUTODOCK (Oxford Molecular Inc.) which systematically searches the conformational space available to the peptide within the antibody combining site. 4) Each positioned conformation is energetically optimized within the antibody combining site using molecular mechanics and molecular dynamics programs that are typically available commercially. Conformational energy calculations (mechanics and dynamics) are performed with the program DISCOVER (Biosym Technologies/MSI) using a variety of force field and solvent parameters. The energy functions and parameters supplied with DISCOVER are well described. Both restrained and unrestrained calculations are performed. Initially, the calculations are performed in vacuo employing a distance dependent dielectric model. Side chains and N and C terminal groups will be defined to be in their neutral state to prevent unscreened interactions between charged groups from dominating the structural search. Typical convergence criteria are assumed for complete optimization.. Molecular dynamics protocols will be those described in our pervious studies. 5) All low energy conformations are tabulated for comparisons. 6) Low energy conformations found in the combining site are again subjected to molecular dynamics calculations (devoid of the antibody and with solvent) to identify differences in binding mode conformations and energies relative to the unbound state. In order to have an estimation of the ‘apparent binding energies’ (which does not include the solvent effect), the same procedure is reproduced with the ligands 50 angstroms away from the antibody combining site to get the energy of the same system, with the same internal degrees of freedom.

In the second phase, binding mode conformations and the conformational properties of putative motifs are further compared with structural information established for respective carbohydrate antigens. These comparisons are tantamount to studies on designing pharmacophores in which charge and spatial relationships are established. Identified peptides from the phage libraries are said to be functionally equivalent and will be compared to find common relationships with Le carbohydrates in structural databases. The computer programs SEARCH and COMPARE (Biosym), and ASP (Oxford Molecular, Inc.), will be used to analyze the known structures. The program SEARCH and COMPARE (Biosym Technologies) which we have used in our modeling of LeY conformations are implemented to evaluate subpopulations of the peptide overlapping with the carbohydrate conformations. Care must be taken in running SEARCH and COMPARE because of the lengths of the peptides Conformational sweeps through angles will be restricted initially to 60 degree differences to explore the general conformational trends. The program ASP (Oxford Molecular Inc.) will be further implemented to define charge and shape relationships. ASP will find common features by considering electrostatic potential, electrostatic field and shape relationships between the peptide and carbohydrate moieties. Common structural features among the isolated phage sequences and analogs, may suggest further motifs that can be used to construct a putative active conformation for further evaluation.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

### Example 1

Antigenic Mimicry of Peptide Motifs

Peptides containing putative aromatic-aromatic motifs have been defined to mimic several carbohydrate subunits (Table 2). These peptides demonstrate the preference of aromatic groups separated by an intervening residue. Peptide motifs identified to mimic these carbohydrate forms are YYPY (SEQ ID NO: 4) as a mimic of mannose as identified from peptide phage screening with Con A, WRY found to mimic α(1-4)glucose as identified from analysis of protein that bind to α-amylase, PWLY (SEQ ID NO: 5) found to mimic LeY as identified from peptide phage screening with an anti-LeY antibody, B3, and YYRYD derived from an anti-idiotypic antibody found to mimic the major C polysaccharide α(2-9) sialic acid (MCP) of Neisseria meningitidis. Aromatic containing motifs have previously been described. In addition aromatic motifs have been isolated from screening a peptide library with anti-polysaccharide antibodies to Cryptococcus neoformans.

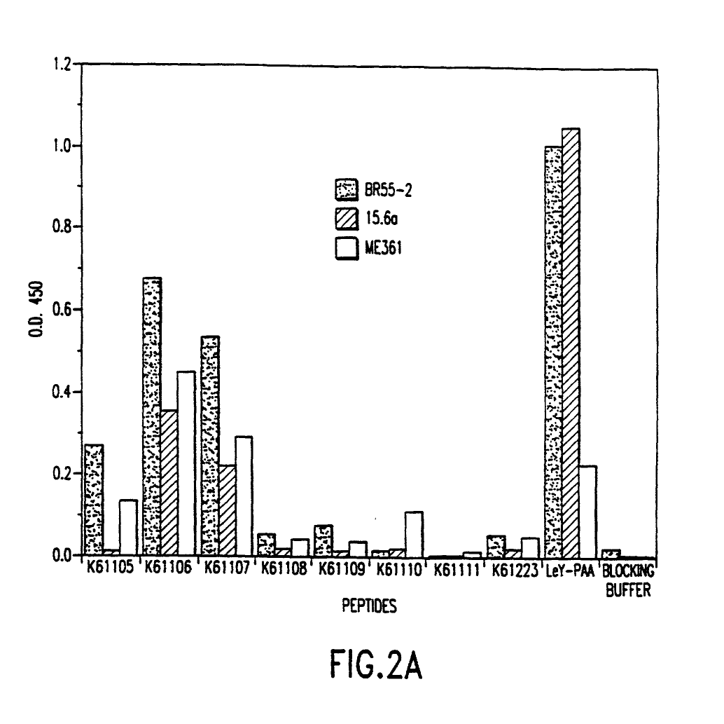

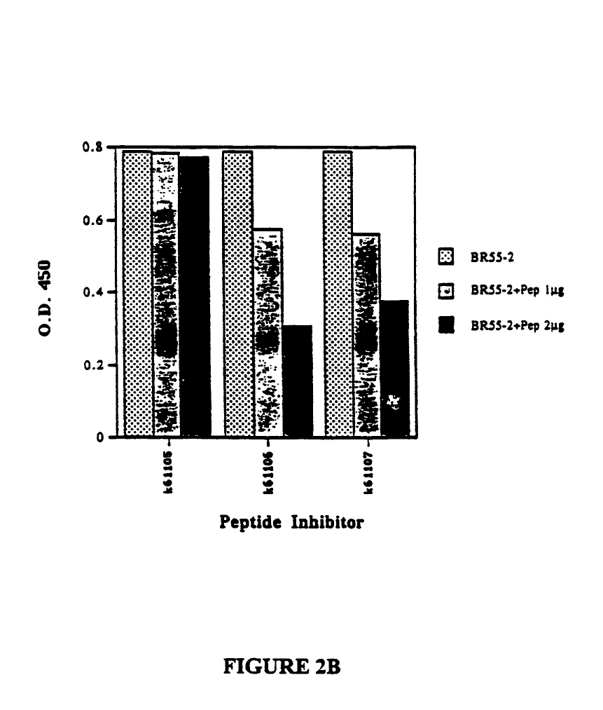

The sequence similarities among the putative motifs suggest that antibodies raised to this peptide set might cross-react with similar subunits expressed on what are otherwise dissimilar carbohydrate structures. For example, polyclonal antibodies raised against the motif YYRYD might cross-react with MCP and with LeY. Molecular modeling suggests that the LeY tetrasaccharide structure is similar to the core structure of MCP, providing a structural basis for potential cross-reactivity . To further determine the extent of cross-reactivity for these motifs, peptides were synthesized repeating the respective putative centralized motifs shown in Table 2. It is theorized that the repeating tract should adopt a helix configuration which emulates many extended carbohydrate structures. To evaluate the antigenic mimicry of motif forms, we synthesized respective multiple antigen peptides (MAP) forms for detection of a reactivity pattern with the anti-LeY monoclonal antibodies BR55-2, and 15.6 (also referred to as BR15.6) and the anti-ganglioside antibody ME361 (FIG. 2a). Significant reactive sequences in FIG. 2a correspond to 3 peptides,

| | |
|---|---|
| GGIYYPYDIYYPYDIYYPYD | (SEQ ID NO:1), (k61105), |
| GGIYWRYDIYWRYDIYWRYD | (SEQ ID NO:2), (K1106) and |
| GGYYRYDIYYRYDIYYRYD | (SEQ ID NO:3), (K61107). |

Other peptides include a triple repeat of the APWLY (SEQ ID NO: 10) motif reactive with another anti-LeY antibody B3 [Hoess, 1993]—e.g. GGGAPWLYGAPWLYGAPWLY (K61223) and a derivatized form were not reactive with these antibodies.

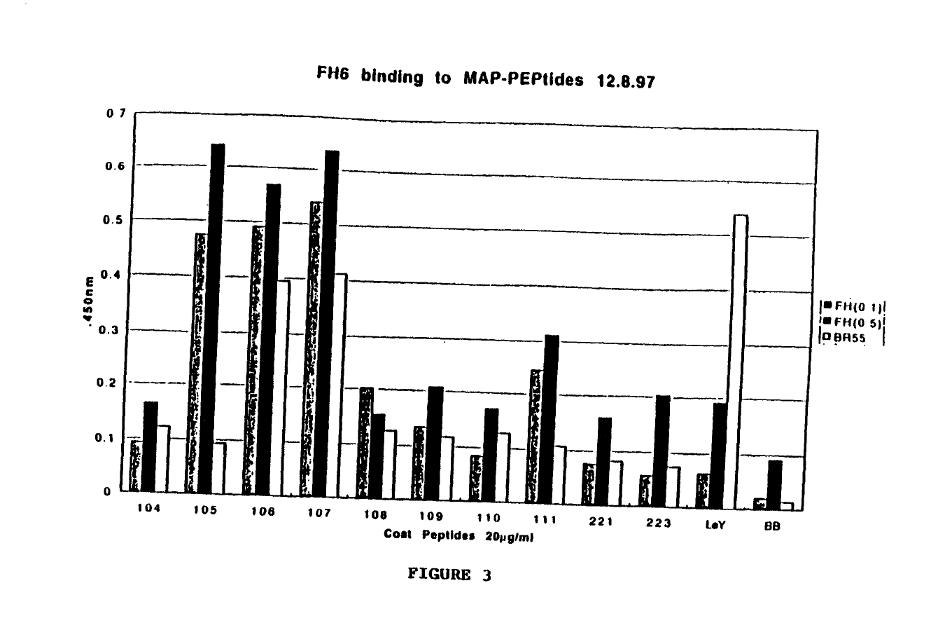

BR55-2 bound very well to K61106 and K61107 relative to the other peptides. Unlike 15.6A, the monoclonal ME361 also reacted with these peptide forms, displaying O.D.s for K61105 and K61110 similar to that observed for the synthetic LeY antigen. The reduction in reactivity of 15.6A for peptides otherwise reactive with BR55-2 suggests that the peptides mimic a structural feature(s) unique to BR55-2 recognition. BR55-2 and 15.6A show distinct binding properties for LeY expressing tumor cells. In addition, BR55-2 displays little reactivity with the K61223 and K61108 peptides which represents the APWLY (SEQ ID NO: 10) motif reactive with the anti-LeY antibody B3 . The affect of sequence on reactivity is observed with lack of reactivity of BR55-2 with K61109 in which the WRY tract was synthesized in a different molecular environment. This data further suggests that K61106 and K61107 mimic salient features of the surface conformation of LeY which is compatible with the BR55-2 combining site since BR55-2 selectively cross-reacts with these peptides. Inhibition of LeY-PAA binding of BR55-2 by these MAP peptides is shown in FIG. 2b. K61106 and K1107 displayed 50% inhibition of BR55-2 binding to LeY with 20 times molar excess. These data indicate that the YRY and WRY motifs synthesized as a triplet, lend to reactivity of these motifs with BR55-2. Substitution of YPY (K61105) reduces the recognition ability of BR55-2. The general mimicry of the K1106, K1105 and K1107 peptides for Le antigens is further assessed by ELISA reactivity with the anti-SA-LeX monoclonal antibody FH-6 (FIG. 3). These peptides are also reactive with this antibody. The significance of this reactivity is that previous reports indicate that peptides might only be reactive with antibodies that isolate them and in fact it is expected that peptide mimotopes might be not true mimics of carbohydrates. Contrary to this suggestion, it appears that the K1106, K1105 and K1107 triple motif peptides mimic a core structure on Lewis antigens, and components of bacterial LOS, that may be further manipulated in a vaccine design effort to target Lewis expressing tumors or pathogens that express these core structures.

### Example 2

The Induction of Anti-carbohydrate Immune Responses by Peptides.

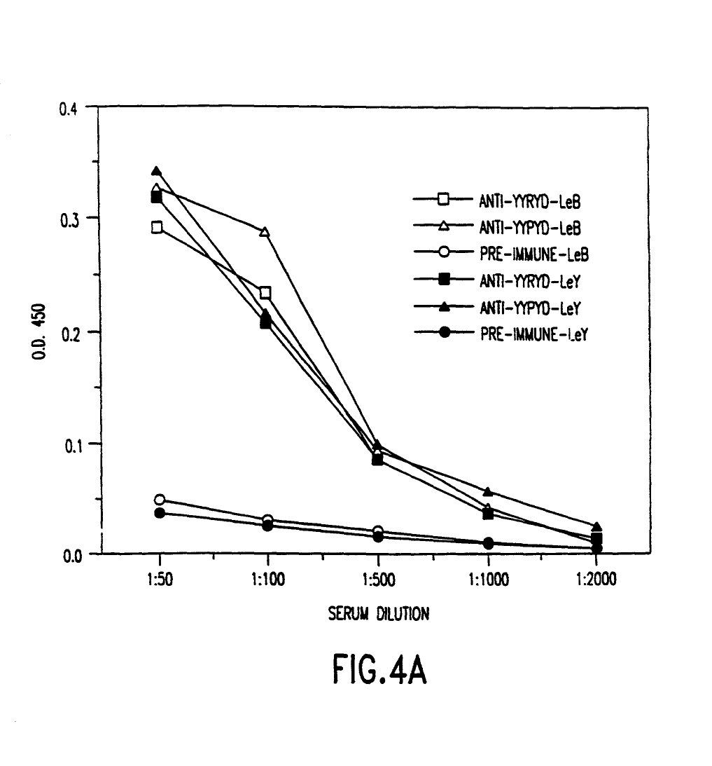

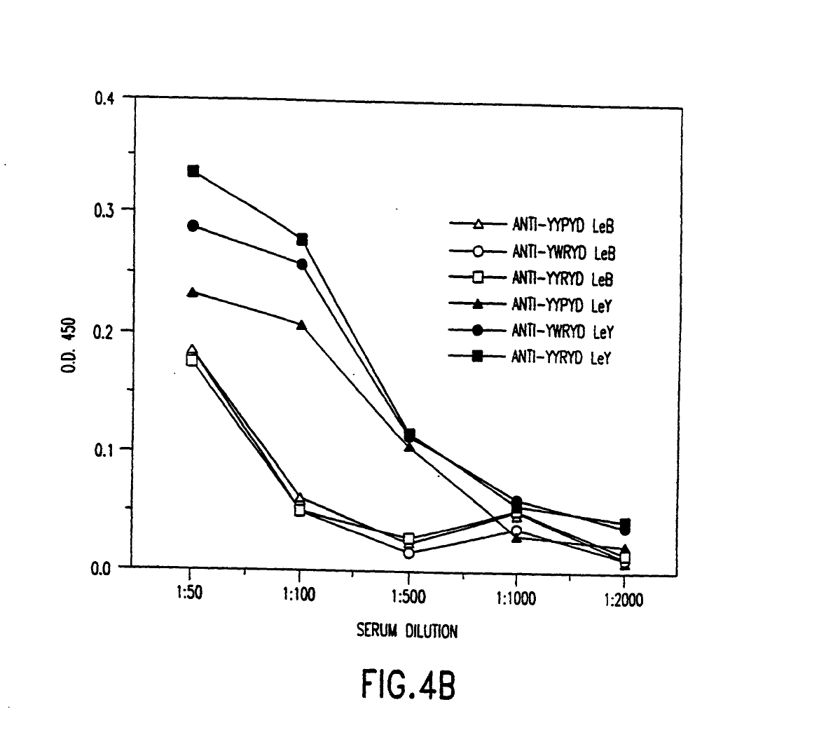

The above mentioned possible structural similarities suggest that anti-sera raised to the peptide putative motifs might cross-react with a variety of subunits representative of Lewis antigens. The immunological presentation of the putative motifs, (i.e. short or longer peptides, presentation in a helix or beta bend, monovalent or multivalent (clustered forms)) might mimic overlapping epitopes on otherwise different carbohydrate structures. To test this idea, Balb/c mice were immunized with peptide-proteosome conjugates representative of the motifs YYPYD (SEQ ID NO: 6), and YYRYD (SEQ ID NO: 7) or the same peptides as MAP forms that are multiple repeats of the motifs administered with QS-21. Sera were collected 1 week after the last immunization, pooled and tested for reactivity with LeY and Leb. For the proteosome conjugates we found that sera developed from the immunizations react with the two multivalent probes, with the IgG reactivity titering up to 1:2000 (FIG. 4a). Superposition of LeY and Leb structures indicate that despite the change of glycosidic linkage from β1-3 to β1-4 in the type 1 and 2 chains, resulting conformational features of the respective sugar moieties are still shared forming a common topography. The only effective difference is the position of the N-acetyl and hydroxymethyl groups projected on opposite sides of the type 1 and 2 difucosylated structures. The ELISA results in FIG. 3a suggest that the sera is reacting with the common topography of LeY and Leb.

Sera derived from immunization with the MAP peptides indicate that the IgM isotype is the predominate form, titering out to 1:2000 for the two carbohydrate forms, but displaying significant differences in reactivity for LeY and Leb that are accentuated at 1:100 and 1:500 titer (FIG. 4b). In contrast to the IgG pre-immune sera, the IgM fraction of the pre-immune sera consistently displayed higher reactivity with the carbohydrate probes, enhancing the non-specific binding.

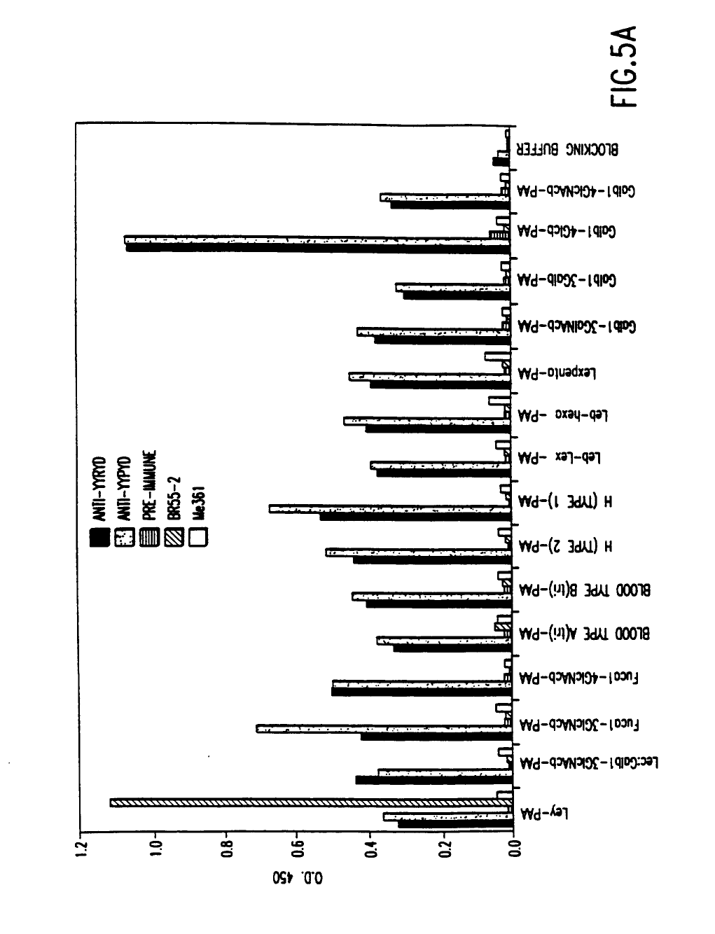

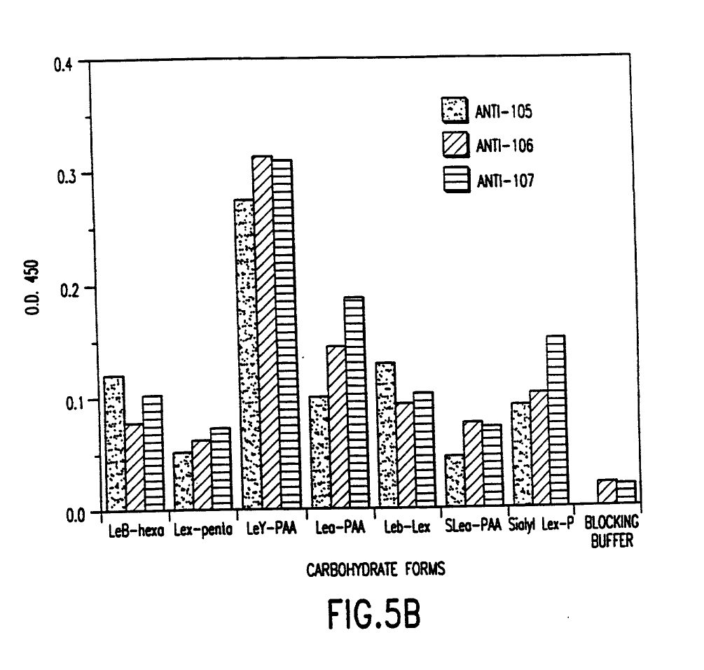

To further evaluate the specificity of the anti-carbohydrate IgG fraction derived from proteosome-conjugate immunized mice, ELISA assays were performed with plates coated with various synthetic carbohydrate subunit probes (FIG. 5a). For BR55-2, selective binding was observed for LeY. ME361 was not reactive with any of the synthetic probes. It was observed that the anti-peptide sera (1:100 titer) was reactive with respective carbohydrate probes above background binding. Preferences for antisera against the YPY motif include Fucα1-3GlcNAc representative of LeY, and Lex, the H type 1 structure Fucα1-2Galβ1-3GlcNAc representative of Leb, and the disaccharide Galb1-4Glc which is a major constituent bacterial LOS. For the YRY motif, reactivity again was observed for all of the synthetic probes, with the disaccharide Galβ1-4Glc displaying the highest reactivity. The antisera reacted with both H type 1 and H type 2 (Fucα1-2Galβ1-4GlcNAc) structures, with the type 1 structure displaying slightly more activity. This may reflect interaction with the common Fucα1-2Gal structure found on type 1 and type 2 structures or functional groups that are shared within the common topography of these two chains. Reactivity was observed for the TF representative subunit Galβ1→3GalNAc, and the Leb and Lea Fucα1→4GlcNAc subunit. These data suggest that there is a large degree of overlap in the potential carbohydrate structures being recognized by the antisera which was expected since the peptides mimic a wide range of singular carbohydrate subunits.

In contrast to the proteosome-peptide conjugates, increased specificity for LeY is observed for the MAP forms (FIG. 5b). At 1:50 dilution, the LeY reactivity of the antisera is approximately 3 fold more reactive with LeY than with Leb, Lex-pentasaccharide, SLea, SLex. Antisera to the YYRYD (SEQ ID NO: 7) containing MAP (K61107) displayed about 1 fold less reactivity with Lea and Sialyl-Lex. Considering the diminished reactivity against LeX which shares the Galβ1→4(Fucα1→3)GlcNAcβ1 component it would appear that a fraction of antibodies react with a conformational component of LeY, providing for its increased reactivity, or alternatively, the affinity for LeY is increased. For either reason, these data suggest that multivalent or multiple antigen peptide forms might provide increased selectivity or avidity for polyvalent or clustered Lewis forms.

### Example 3

Distribution of Sera Reactivity

An important consideration in the development of synthetic surrogate immunogens is the reactivity of the induced IgG sera to react with naturally expressed carbohydrate on the tumor surface. We have evaluated the ability of IgG elicited by peptide-proteosome, compared with IgM fractions induced by MAP forms of the same peptides, to bind to representative tumor cells as evaluated by FACS assay (Table 3). Positive control monoclonals were BR55-2 and ME361. Normal mouse sera (NMS) and sera generated against proteosome alone were also used as a controls. Of interest was whether induced predominant IgG or IgM sera react the same. We found that sera from MAP peptide immunized mice displayed a higher mean fluorescence for MCF7 cells than the proteosome formulation. For SKBR3 cells both sera types reacted about the same. Both sera types displayed minimal reactivity with the normal breast cell line and murine fibroblast. Anti-YYRYD and Anti-YYPY sera reacted with WM793 cells but to different degrees. Melanoma cells have recently shown to express sLeX and sLea. We tested the WM793 cells by FACS with the antibody FH-6 which is specific for sLeX. Reactivity was observed for this antibody. Our results suggest that the respective sera is perhaps cross-reacting with sLeX on the WM793 cells since we see some reactivity with this probe. This data suggests that even though this sera reacts with common structural features of MCP and LeY, the presentation of related carbohydrate forms on tissues is affected by the carrier molecules to which they are attached or the density of expressed carbohydrate is low. Saito has described effects of carrier molecules.

### Example 4

Carbohydrate Modification Affects Sera Reactivity.

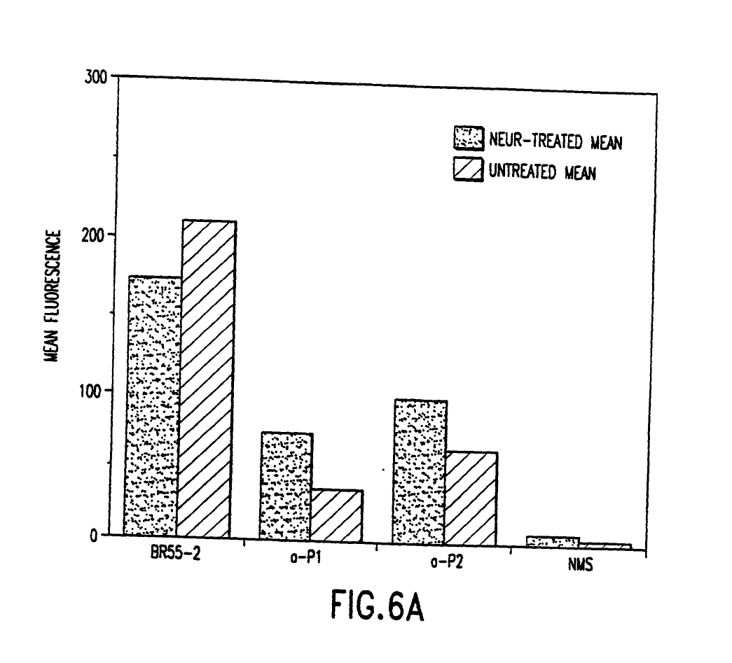

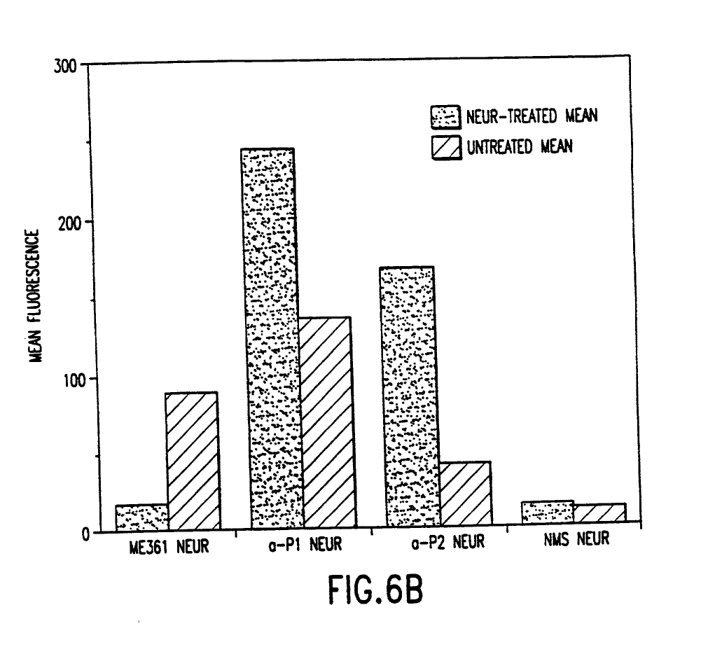

The ability of the peptides to mimic carbohydrate fragments or subunits on the cell surface is further observed from consideration of treating cells with neuraminidase and then letting the sera react with the cell (FIG. 6). Treating SKBR3 cells with neuraminidase marginally decreased the anti-LeY BR55-2 antibody binding to the cells, while marginally increasing reactivity with the peptide antisera (FIG. 6a). This result indicates that some of the carbohydrates are sialylated (i.e.STn) on the SKBR3 line and their removal may affect the conformational properties of some carbohydrates, exposing new epitopes recognized by the antisera. Treatment of the WM793 human melanoma line with neuraminidase significantly decreased ME36 1 recognition of these cells, consistent with the recognition of sialyl subunits on the GD2/GD3 antigen (FIG. 6b) Significant increases in the mean fluorescence was observed for antisera binding to WM793 cells. The increase of binding of the anti-sera is interpreted as exposing otherwise encryptic epitopes on the cell surface after sialic acid removal. The core structure for GD2 is GalNAcβ1→4Gal[3←2αNeuNAc8←2αNeuNAc]b1→4Glcβ1→1 Cer and for GD3 (NeuNAcα2→8NeuNAcα2→3Galβ1→4Glcβ1→1Cer). Presumably, elimination of sialic acid results in exposure of GalNAcβ1-4Gal units associated with GD2 and Galβ1-4Glc units associated with GD3. Representative synthetic probes of these subunits are highly reactive with the antisera (FIG. 5a). This data further suggests that carbohydrates on the surface of tumor cells are seen by the anti-peptide sera.

To further determine the immunochemical characterization of cell surface carbohydrates, we have performed immunoprecipitation of tumor cell lysates before and after treatment with tunicamycin. LeY epitopes are found to be expressed on MUC-1 mucins, lower molecular weight glycoproteins and glycolipids, as well as higher m.w. proteins like CEA and LAMP-1. BR55-2 immunoprecipitates (IP) neoproteins in the range >200,000 <43,000 found on SKBR3 cells. This profile is similar to that observed for anti-LeY antibodies B1 and B3, and BR96. IP with NMS indicates no reactivity within this molecular weight range. Treatment of SKBR3 cells with tunicamycin for 2 hrs decreases neoglycoprotein reactivity with BR55-2, verifying the carbohydrate recognition of this antibody. IP of SKBR3 cells with the anti-peptide sera indicates that the P1 and P2 anti-sera display an IP profile similar to that of BR55-2. Strong reactive bands for P1 and P2 are in the range around 47 to 89KD, with weaker bands between 117 KD and 89 KD. These IP bands correspond to LAMP-1 reactive KDs originally identified with the anti-LeY monoclonal antibody BR96. We found that treatment of cells with tunicamycin for 2 hrs. decreases carbohydrate expression of neoglycoproteins reactive with the anti-sera. However, unlike BR55-2 reactivity, the respective sera is reactive with suspected glycoproteins around 47 KD after 2 hrs. Bands around the 47 KD region are identified by BR55-2 prior to neuraminidase treatment. It is possible that our sera is reacting with human breast carcinoma antigen BA46 since immunoprecipitation profiles indicate a protein at 46 KD. This protein contains an RGD tract in its EGF-like domain. We observed however that antisera directed toward the YYRGD (SEQ ID NO: 8) motif does not immunoprecipitate any of the glycoproteins in the cell lysates pre and post treatment. This data further suggests that the anti-sera is reactive with carbohydrate epitopes on the cell surface which is similar to that observed for anti-LeY monoclonal antibodies.

### Example 5

Tumor Cell Cytotoxicity

Anti-carbohydrate antibodies may mediate complement dependent cytotoxicity (CDC) better than cytotoxicity associated with various effector cells. Subsequently, we have initially examined CDC mediation of various sera raised either to peptides or against the multivalent LeY-PAA form. This form has been shown to be immunogenic in mice when adsorbed onto bacteria (Salmonella minnesota). We examined the ability of the sera to mediate complement dependent cytotoxicity (CDC) of the SKBR3 and MCF7 human breast adenocarcinoma cell lines, the ovarian tumor cell line OVAR-3 and the human melanoma lines WM793 and SKMEL-28 compared to that of the LeY-PAA generated sera. Positive control antibodies were BR55-2 which mediates CDC of the adenocarcinoma lines, and ME361 which mediates killing of WM793. Negative controls were pre-immune sera, an irrelevant peptide immunogen (C1) and two peptides (G1 and G2) isolated from phage display that are reactive with ME361.

Peptides P1, and P2 both showed an ability to mediate CDC of the LeY expressing human breast lines SKBR3 and the human ovarian line OVAR-3 similar to the positive control BR55-2 MAb. Sera raised against P2 showed diminished CDC activity for MCF7. P2 mediated CDC of the human melanoma lines close to non-specific values using control sera (C1), while P1 displayed moderate CDC activity. The differential CDC activity for the adenocarcinoma and melanoma cells by P1 and P2 antisera are also reflected in the displayed CDC activity mediated by antisera to the G1 and G2 peptides. Data indicates that the functional response can be specific for carbohydrates highly expressed on human tumors. The P2 reactive sera displays a clear preference for the adenocarcinoma cells, while P1 reacts slightly more with the ganglioside expressing cells. These data also suggest that despite the broad specificity of the sera for carbohydrate constituents by ELISA, the respective sera recognize ubiquitous carbohydrate subunits differently when expressed on cells. These data subsequently indicate that sera generated to carbohydrate mimicking peptides have the potential to recognize important tumor associated antigens with a high degree of specificity.

### Example 6

Neutralization of HIV

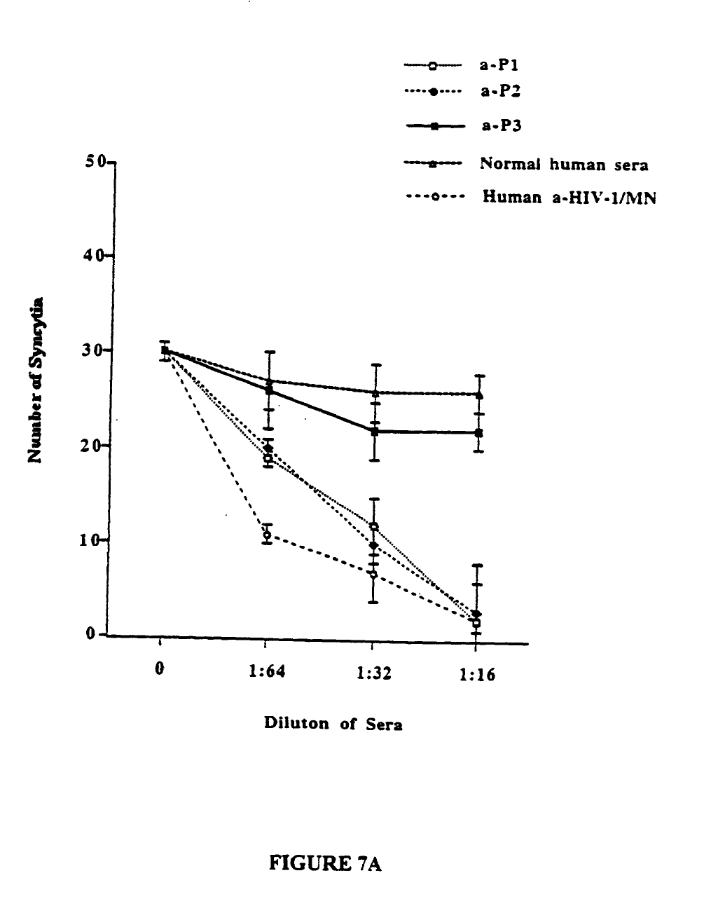

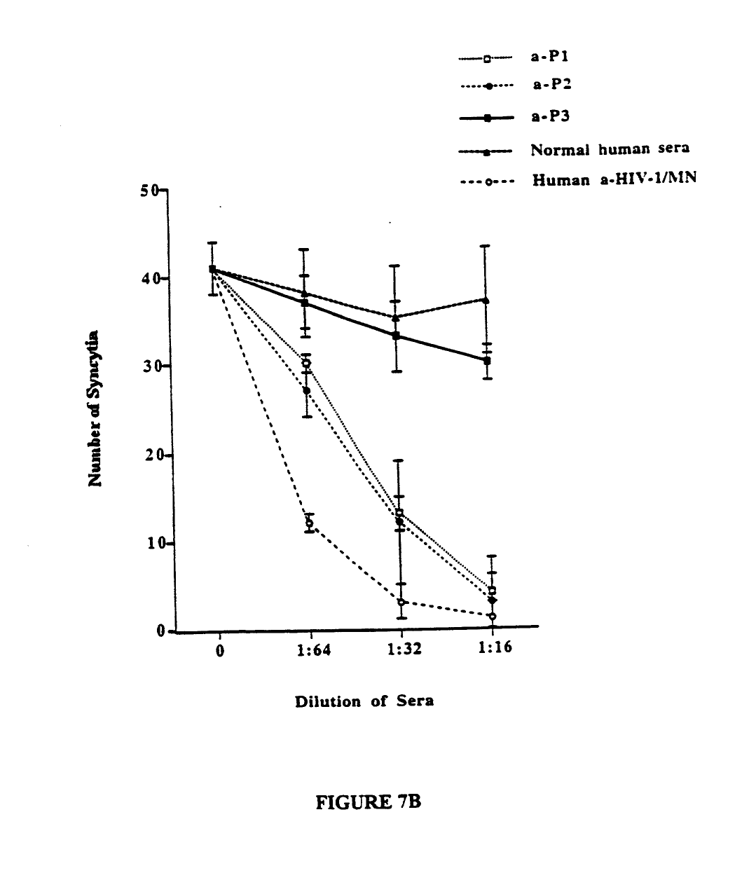

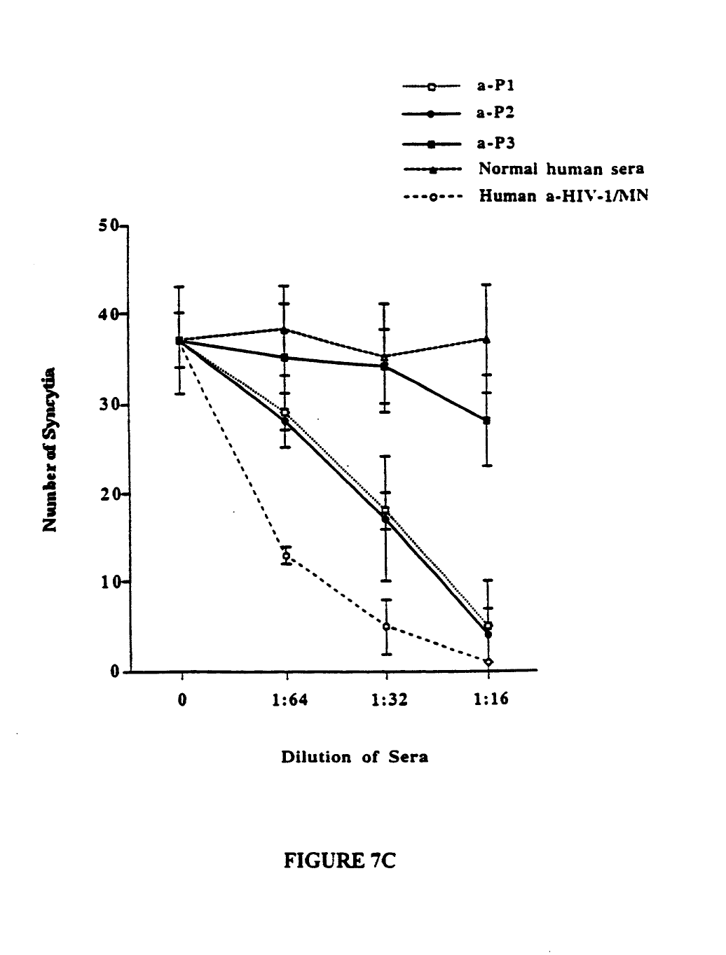

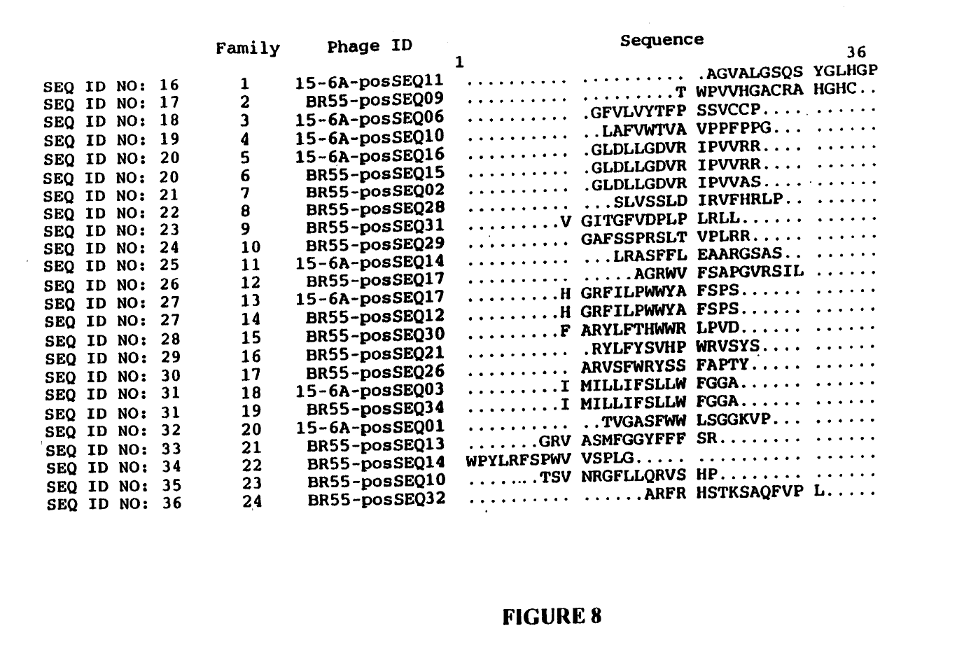

Mannose, sialyl and histo-blood group related carbohydrate epitopes (particularly LeY) are found on the major envelope protein of the human immunodeficiency virus (HIV). Consequently, we examined the extent to which the anti-peptide sera could mediate HIV-1 cell free neutralization. We observed that sera from Balb/c and C57B1/6 mice immunized with P1 or P2, neutralized HIV-1/MN at final dilution's up to 1:64 (FIGS. 7a-c). FIG. 7a illustrates the neutralization of HIV-1/MN by anti-P1 and P2 sera from Balb/c mice. Normal human sera (NHS), as well as the anti-P3 sera, were ineffective at blocking syncytia formnation, whereas human α-HIV-1 sera from 4 different infected patients neutralize cell free virus at dilution's up to 1:64. Sera derived from C57B1/6 mice also followed this profile (FIG. 7b). The isolate specificity was further determined by cell free neutralization of the HIV-1/3B isolate (FIG. 7c). As with MN, anti-P1 and P2 Balb/c sera were effective in neutralizing virus infection in vitro. Collectively, these results suggest that the production of HIV-neutralizing antibodies by the peptide-proteosome complexes induces humoral immune responses in divergent haplotypes that can be as effective as sera from HIV-1 infected individuals in neutralizing HIV-1 cell free infection.

### Example 7

Placement of a Peptide Mimotope into Anti-LeY reactive Antibody Combining Sites.

To further design peptide mimotopes of carbohydrate antigens, molecular modeling that establishes quantitative structure activity relationships can be used. The crystal structure of BR96 provides a template to directly model the homologous anti-LeY B3 and BR55-2 structures. These antibodies share a high degree of sequence homology with BR96 in both their light and heavy chains. Potential hydrogen bonding sites between *BR96 and the tetrasaccharide core structure of LeY are observed to be highly conserved between BR96 and B3 and BR55-2. Potential contacts are based upon heavy atom distances measured in the BR96 structure. This information allows for placement of the LeY antigen into the B3 and BR55-2 combining sites.

The B3 antibody binds to the peptide sequence APWLYGPA (SEQ ID NO: 9) presented on phage display in which the putative pentapeptide sequence APWLY (SEQ ID NO: 10) is critical for binding to the antibody. To establish how this putative sequence mimics LeY binding to B3, we fit the pentapeptide sequence into the B3 combining site using the program LIGAND-DESIGN (LUDI, Biosym Technologies) (Bohm, L. W. et al., J. Mol. Recogn., 6:131-137 which is incorporated herein by reference). This program searches a molecular library for fragment fs representative of the amino acids in the target peptide sequence. The program then positions the fragments within the combining site devoid of steric conflicts. The APWLY (SEQ ID NO: 10) sequence was modeled such that the Trp, Tyr, Leu and Ala residues occupied relative positions as the identified LUDI fragments. The judicious positioning relied upon intermolecular interaction calculations in which several potential binding modes of the peptide were ranked according to the stability of the complex. In the most stable conformation, we observed that the AP residues occupied a similar position to the LeY GlcNAc residue. This positioning indicates that the proline residue mimics the spatial position of the glucose unit of GlcNAc, while the Ala methyl group is positioned similarly as the terminal methyl group of GlcNAc's—acetyl. The Trp residue occupies a volume associated with the Fuc 1-3 moiety, and the Leu residue occupying the volume and hydrophobic interaction of bGal. The Tyr residue occupies a position not associated with LeY binding to B3. We observe that the low energy binding mode conformation adopts a turn region similar to that observed for the YPY motif in binding to ConA. This conformation lends itself to the Tyr residue of the peptide to potentially interact with several residues in CDR2 of the heavy chain of B3 that include Asp H53, Ser H52, Ser H55, or Ser H56. These residues are different in BR55-2, which does not bind the monovalent APWLYGPA (SEQ ID NO: 9) peptide in a series of ELISA assays. Energy optimization of the positioned peptide identified similar functional groups within the B3 combining site in contact with the peptide and carbohydrate tetrasaccharide core of LeY. This analysis therefore defines a strategy for determining the molecular basis for antigenic mimicry of particular motifs, providing a unique perspective of how a peptide sequence fits into the antibody combining site, competing with a native antigen.

In the placement of the B3 reactive putative peptide sequence APWLY (SEQ ID NO: 10), we made use of the program LUDI to identify compounds that potentially interact with the B3 combining site. Over 260 fragments were identified for the model, with the largest radius of interaction, with most redundant for the same set of potential hydrogen bond donors or acceptors on B3. In evaluating the fragments we compared fragments identified by LUDI relative to the APWLY (SEQ ID NO: 10) sequence such that the fragments could occupy non-redundant sites and be spatially far enough from each other to accommodate the peptide backbone. In FIG. 3 the placement of representative LUDI fragments is shown relative to their positions with each other within the B3 binding site of the respective models. LUDI found that a Trp like residue forms a hydrogen bond with the backbone carbonyl oxygen of Trp H98, that a lipophilic residue representative of a Leu side chain is bounded by residues Val L94, His L27D, and Ala H58 another lipophilic residue representative of an Ala and Pro side chain is bounded by Ala H97.

The APWLY (SEQ ID NO: 10) sequence was then modeled such that the corresponding Trp, Pro, Leu and Ala residues occupied relative positions as the identified LUDI fragments. In affect one wants to “stitch” the fragments together to form a peptide. We modeled the peptides two ways. The first, was to use individual amino acid fragments oriented with their side chains superimposed on the LUDI identified side chain types. The individual fragments were then restrained to form concomitant backbone geometry's and conformations. As expected, such an approach resulted in highly strained conformations. Alternatively, a peptide was built and the phi, psi angles rotated until the respective side chains were in close proximity. The positioned peptide fragment-B3 complex was then energy optimized with a restrained dynamics calculation. After this dynamics run, the complex was again energy optimized to convergence without the imposition of constraints. Deviation of the backbone conformation of the peptide-B3 complex relative to the respective LeY-B3 complex was found to be only 0.29 A. This indicates that the placement of the peptide within the antibody combining site did not dramatically alter the overall conformation of either B3 structures.

While the LUDI search provided a favorable geometry for peptide side chain placement, the final placement of the peptide side chains within the antibody combining site relative to the LUDI positioned fragments were different. Several different starting geometry's for the peptide placement in the BR96 model were tested. Intermolecular interaction calculations indicate that the majority of the peptide binding comes from dispersion interactions. Five potential hydrogen bonds were found for the most stable of the models. One involves the N7 of Trp interacting with the backbone carbonyl group of Trp H98, the carbonyl backbone of Trp interacting with His L27D, the Tyr side chain hydroxyl group interacting with hydroxyl group of Ser H55, the backbone carbonyl group of Ala interacting with Asn H52A, and Tyr H33 side chain interacting with the carbonyl backbone of Leu, whose hydrophobic side chain being fuirther stabilized by dispersion interactions with Val L94. We have further constrained the model peptide to form a beta turn in which a hydrogen bond is potentially formed between Tyr amide and Pro carbonyl groups. This is a low energy conformation for this structure. This structural template may be useful un the development of further designs both for vaccines and for the development of inhibitors that react with carbohydrate receptors.

### Example 8

Peptides Reactive with Anti-LeY and Ganglioside Antibodies

We extended our approach described above to determine what kind of motifs could bind to BR55-2 and whether we could isolate such motifs with BR55-2 from a peptide phage screen. If we could identify motifs this way then we may be able to improve upon the antigenic mimicry for LeY. We know YPY, YRY and WRY motifs are mimics of various carbohydrate subunits. Searching the LUDI database using the modeled structure of BR55-2, we identified these motifs as interacting with BR55-2. In the LUDI search, we also identified a non planar-X-planar type motif, FSLLW (SEQ ID NO: 11), as a possibility.

We next screened a 15 mer peptide library with BR55-2 and BR15-6A. The initial choice of using the 15 mer library was predicated on the notion that this length is similar to complementarity determining regions (CDR) in antibodies which confers mimicry capacity to many anti-idiotypic antibodies. We have sequenced isolated phage reactive with BR55-2 and 15-6A (FIG. 5). These sequences can be compared with the putative peptide identified to bind B3. We have tested the APWLYAP (SEQ ID NO: 12) peptide for binding to BR55-2 and BR15-6A which proved negative. Nevertheless, the central Planar residue-X-Planar residue tract is represented in families 12, 14-17, 21 and 22 for BR55-2 and observed in families 3, 4, 13 and 20 for 15-6A. In family 22, identified by BR55-2, an inverted tract WPYL (SEQ ID NO: 13) is observed in comparison to the sequence PWLY (SEQ ID NO: 5) found by B3. A sequence tract of WRY associated with a(1-4) glucose structure is observed in Family 17 of BR55-2. The above sequences were multiply aligned using the program Pileup (FIG. 5). We observe that three groups 5&6, 13&14 and 18&19 are equivalent for the two anti-LeY antibodies, suggesting that perhaps these sets might be idiotypically connected since the two antibodies bind to Lewis Y in a non-competing fashion.

We have also attempted to de novo design a peptide to selectively react with BR55-2 using the approach above to place a peptide in the B3 combining site. In our initial attempt we identified a sequence tract FSLLW (SEQ ID NO: 11) as a possibility. We observed such a tract in families 18 and 19 for BR55-2 and for 15-6A. This result indicates that we can define residues and peptides de novo that are equivalent to those that identified experimentally. It is anticipated that this type of analysis can be used to optimize peptides that are selective for particular antibody subsets, as well as defining peptides that are better mimics of CHO epitopes.

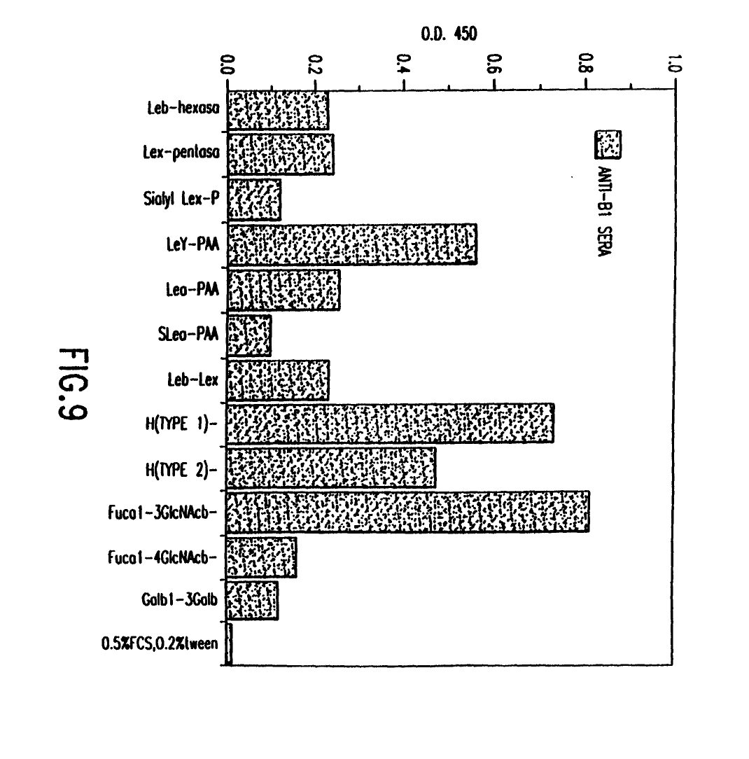

This FSLLW (SEQ ID NO: 11) peptide (#18) (referred to as 1104) is specific for BR55-2 since it does not react with FH6 (FIG. 3). We showed that sera elicited by the proteosome form of this peptide specifically mediated CDC (Table 4). To further evaluate the specificity of the 1104 peptide we synthesized 1104 as a MAP form and immunized Balb/c mice, with QS-21 as adjuvant and examined its reactivity pattern with various Le synthetic probes by ELISA (FIG. 9). The utility of MAP peptides is in their apparent advantage as immunogens. For example, MAPs have proved to retain all the immunological properties of an intact anti-id upon which the peptide was based, and was found to be qualitatively similar and quantitatively superior to the linear monomeric 15 mer anti-Id derived peptide. However, the MAPS described above contain T cell epitopes within the peptide sequence. MAPS that do not contain T cell epitopes do not induce significant IgG titers. We have observed this trend with MAP peptides.

The anti-1104 sera was predominately of IgM isotype as observed in our previous studies. The sera displayed a three to fold increase in reactivity for LeY over Leb, tittering up to 1:2000 in ELISA. At 1:50 serum dilution (FIG. 9), we observed about the same level of reduced reactivity for Leb hexasaccharide, LeX-pentasaccharide, sLeX, Lea, and sLea, while higher levels of reactivity are observed for LeY and LeY constituents. Minimal binding is observed for a ubiquitous disaccharide unit Galβ1-3Gal. This lack of reactivity is in contrast to that observed with sera generated to our general aromatic-aromatic motifs . The best reactivity is observed with the LeY constituent Fucal-3GlcNAc. The anti-1104 sera appears to recognize both Fuc moieties on LeY since LeX displays diminished reactivity. The anti-1104 sera distinguishes the Fucα1-3 and 1-4 GlcNAc linkage, displaying significantly reduced reactivity with Fucα1-4GIcNAc. This selective interaction sets apart reactivities between Leb and LeY, since reactivity is observed for the H type 1 constituent of Leb. These results suggest that we can develop peptides theoretically and by phage isolation that rendered as clustered MAP peptides, are powerful immunogens displaying the desired specificity for tumor antigen by interacting with a specific epitope.

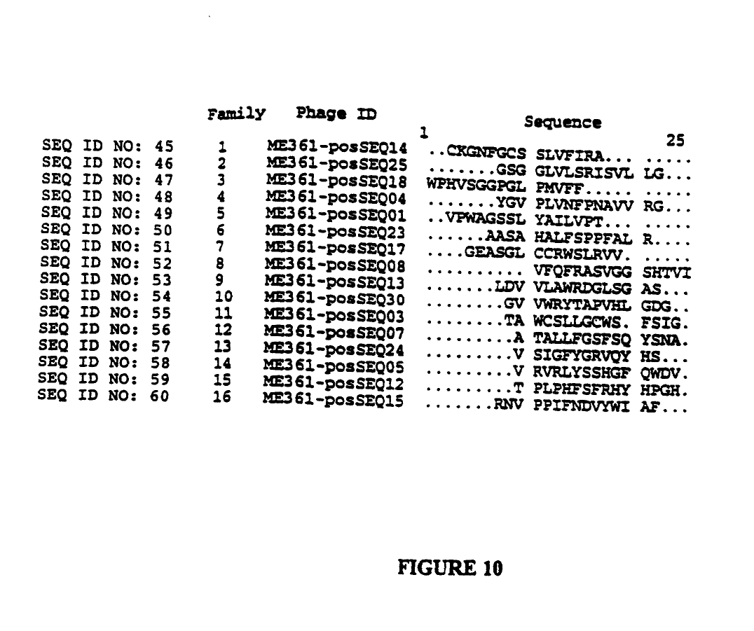

To better define peptide motifs that mimic GD3/GD2, we have screened the 15mer phage library against ME361 and isolated phage were sequenced (FIG. 10). Monoclonal antibody ME36.1 recognizes the GD2 and GD3 ganglioside and shown potential for use in immunotherapy. We observe some relationships between ME361 reactive peptides and BR55-2 and BR15-6A peptides, but for the most part they are separate, indicating that many peptide forms mimic the respective carbohydrate antigens. We observed two sequence tracts representative in the ME361 isolated peptides which are similar to our lead peptides. These include the WRY tract in Family 10, and a WRDG (SEQ ID NO: 14) tract (similar to YRGD (SEQ ID NO: 15)) in family 9, which suggest that the lead peptides mimic carbohydrate subunits found on a variety of carbohydrates explaining the cross-reactivity of the P1-P3 anti-sera for both breast and melanoma cells.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 9

DNA Immunization With Minigens To Induce Anti-Carbohydrate Immune Responses Abstract

Polysaccharide vaccines to encapsulated bacteria and some tumors are weakly immunogenic due to their T-independent nature. Even when converted to T-dependent forms through conjugation to foreign proteins, polysaccharides induce responses that are deficient in many respects, such as induction of T helper 1 (Th 1) associated murine IgG2a antibody. Anti-carbohydrate Th1 immune responses might be particularly useful for the development of pathogen and tumor vaccines. To augment Th1 immune responses to carbohydrate antigens we describe results of DNA vaccination studies in mice with plasmids encoding peptide mimotopes (minigens) of the neolactoseries antigen Lewis Y (LeY). In contrast to native polysaccharide vaccine, immunizations with the glycotope-encoded plasmids induce LeY cross-reactive IgG2a antibodies. The IgG2a immune response persists for at least 2 months. Immunization induces a LeY specific memory response that is rapidly activated upon encounter with nominal antigen upon subsequent boost.

Introduction

Targeting carbohydrate antigens associated with many pathogens or expressed on tumor cells is a challenge in the design of effective vaccines. Most carbohydrate or polysaccharide (PS) immunogens belong to the category of T cell-independent (TI) antigens that reflect their inability to stimulate MHC class II dependent T cell help. Antibodies (Ab) produced in response to vaccination with carbohydrate antigens usually are not of high affinity compared to those produced by responses to peptide or protein antigens. Consequently, carbohydrates do not induce substantial anamnestic or secondary immune responses and therefore require extensive adjuvanticity to recruit T cell help. Conjugate vaccines, in which the carbohydrate or PS antigen is covalently attached to an immunogenic carrier protein is able to overcome the lack of a T helper cell response and can induce higher titer IgM antibodies and partial class switching to IgG antibodies. Even when converted to a T cell-dependent (TD) form, polysaccharides induce responses that are deficient in many respects. These deficiencies include the induction of the T helper 1 (Th1) associated murine IgG2a isotype, resulting in a requirement to overcome this limitation.

An alternative approach to develop T-dependent type responses to carbohydrate antigens, is the use of peptide or polypeptide surrogates that mimic carbohydrate. It has been suggested that carbohydrate mimicking peptides could revolutionize vaccines against infectious pathogens. Peptides that mimic carbohydrate structure have significant advantages as vaccines compared with carbohydrate-protein conjugates. First, the chemical composition and purity of synthesized peptides can be precisely defined. Second, the immunogenicity of the peptides can be significantly enhanced by polymerization or addition of relatively small carrier molecules that reduce the total amount of antigen required for immunization. Third, peptide synthesis may be more practical than synthesis of carbohydrate-protein conjugates. Finally, we propose that peptide mimotopes can be engineered into DNA plasmids for DNA vaccination to further manipulate both B cell and T cell responses.

We have developed peptides that induce immune responses to carbohydrate structures with in vivo and in vitro functionality. We have found that peptides representative of a W/YXY sequence motif can antigenically and immunologically mimic the meningoccal group C capsular polysaccharide (MCP) of Neisseria meningitidis, Lewis (Le) antigens expressed on tumor cells and mannosyl, lactoseries and sialyl-residues on HIV-1 gp 120. Immunization of mice with peptide fomulations induces IgM and IgG carbohydrate cross-reactive responses. We hypothesize that the utility of peptide mimotopes when formulated into a DNA based vaccine (minigen) could potentially increase the breath of the immune response, redirecting the response to include the induction of Th1 mediated mechanisms associated with the clearance of both pathogens and tumor cells alike.

The induction of a Th1-like response is the predominant response to DNA vaccines. A Th1 response with the expression of the IgG2a isotype is a desirable response for many pathogens and for targeting tumors as IgG2a antibodies are opsonizing and complement fixing, and IFN-γ can function to augment pathogen clearance and tumor cell killing. Immunization with peptide mimotope encoded DNA can therefore provide a cellular associated response to carbohydrate antigens that is not achievably by PS-conjugates alone. Redirection of the immune response to a Th1 like profile-targeting polysaccharides of encapsulated bacteria or tumor associated carbohydrate antigens may augment vaccine responsiveness to these otherwise challenging antigens. Here, we report on the feasibility of peptide mimotopes constructed into DNA plasmids for the induction of an anti-carbohydrate immune response.

### Results

Choice of peptides as DNA immunogens

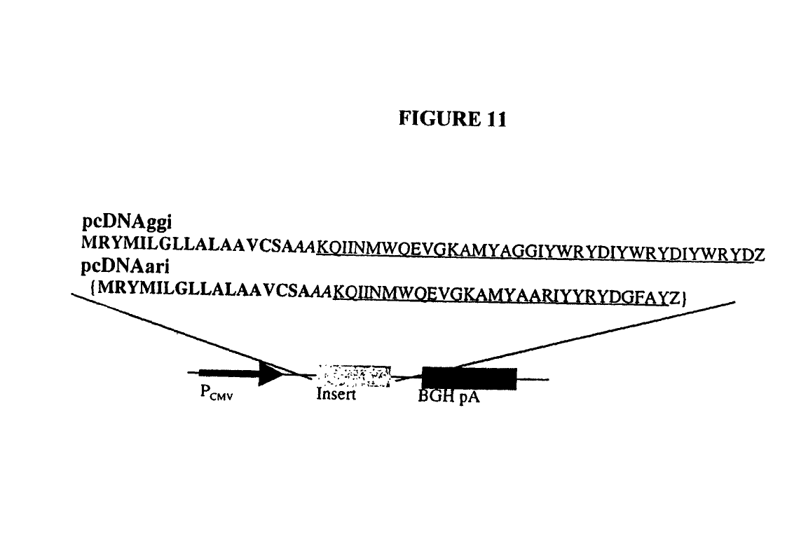

In previous studies we had showed that WYXY (SEQ ID NO:61) containing peptides can induce cross-reactive immune responses to MCP and to the neolactoseries antigen Lewis Y (LeY). The basis for this cross-reactivity was shown to be structural in that LeY and the sialic acid structure of MCP are similar suggesting that antiserum to peptides containing the YRY and WRY sequence tract may also bind to homologues of these structures. To evaluate the potential carbohydrate cross-reactive immune response to these carbohydrate antigens we designed inserts (FIG. 11) for introduction into plasmids for immunization. Plasmids pcDNAari and pcDNAggi were designed to express chimeric peptides that include a secretory leader sequence and the inclusion of the T1 T cell epitope form HIV-1 gp120 with the respective peptide sequences. The peptide sequences in the designed plasmids have been shown to induce cross-reactive humoral responses to both LeY and MCP either conjugated to proteosomes or as multivalent antigenic peptides (MAPs). It was rationalized that the leader sequence would allow exogenous expression with the inclusion of the T cell epitope driving T cell help.

The T1 epitope is suggested to bind to both MHC Class II and Class 1, inducing T helper responses and cytotoxic T lymphocyte (CTL) responses. Potential Class I MHC binding sequences can also be subsets of MHC Class II binding peptides. Representative HLA binding prediction calculations identifies an H2-D (Balb/c) Class II and Class I binding motif centered on the sequence of the T1 peptide (Table 5). Likewise, the sequences are predicted as Class II and Class I binding peptides. In related studies immunization with these sequences as synthetic peptide leads to T cell proliferation that is inhibited by anti-MHC Class II antibody. This finding is indicative that the peptides are processed in association with Class II presentation and T cell activation.

Anti-carbohydrate Reactivity:

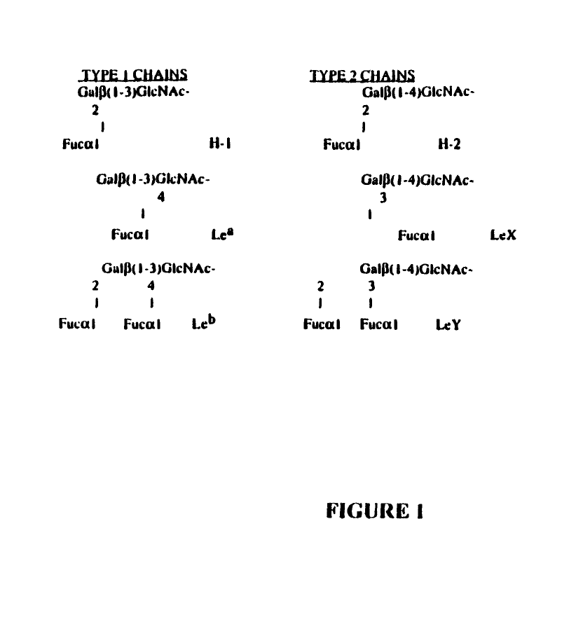

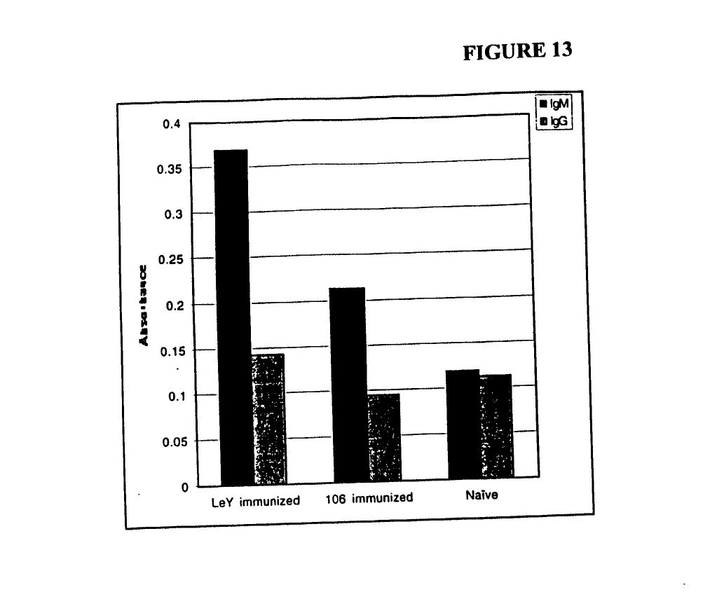

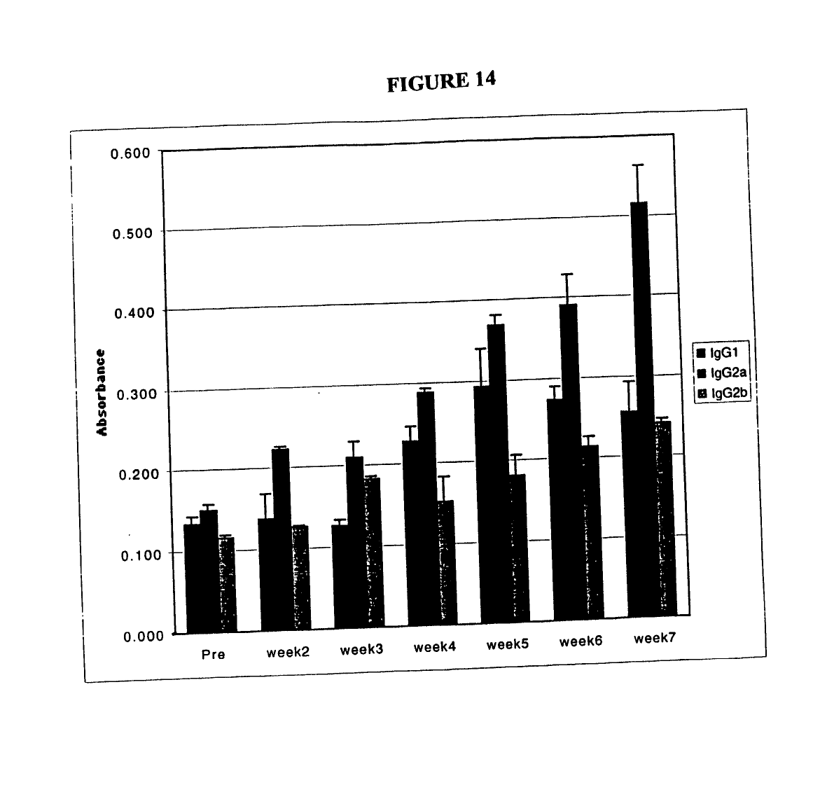

We immunized groups of animals with all constructs and 3 weeks after the immunization we checked serum reactivity with LeY (FIG. 1 2A). Results with ten times diluted sera showed that a significant increase in the level of cross-reactive IgG antibody after immunization with all constructs. In these assays serum reactivity of all DNA constructs peaks within two weeks of boosting the animals. We immunized the mice again at week 4 and determined the titration of anti LeY immune response in immunized mice at week 6 (FIG. 2B). Results show a cross-reactive immune response persistent to a 1:256 dilution, compared to control vector immunized mice, in the serum from pcDNAggi immunized animals. As indicated, immunization with PcDNAggi plasmid, compared to the other two plasmids, induces a higher titer cross-reactive response to LeY. PcDNAggi plasmid, immunized group with a significantly higher antibody titer in the serum sample was selected to further reveal the isotype components of the cross-reactivity. Importantly, the response had been switched to a predominately IgG2a (FIG. 13) isotype. This is in contrast to multivalent polysaccharide or multivalent peptide immunization that induced predominately the IgM isotype (FIG. 14). This switch in isotype is likely the result of the Th 1 bias of the now converted DNA immunogen.

DNA Vaccination Primes for Higher Antibody Response

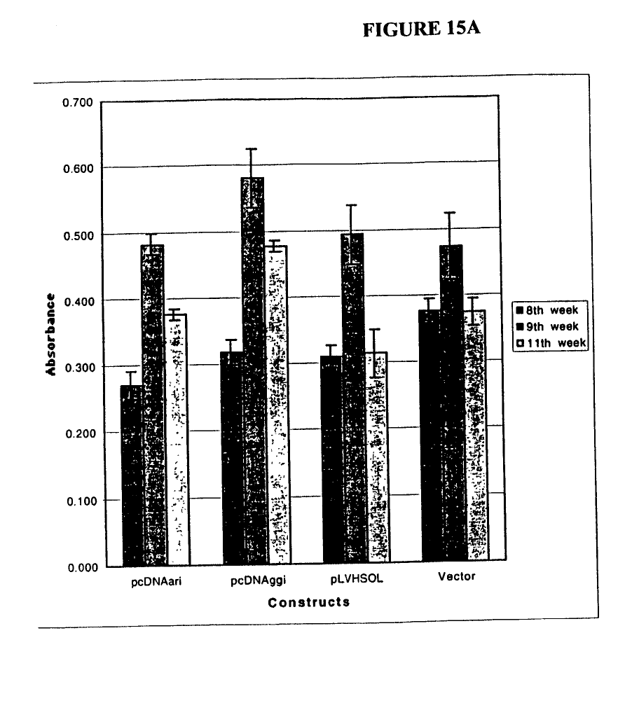

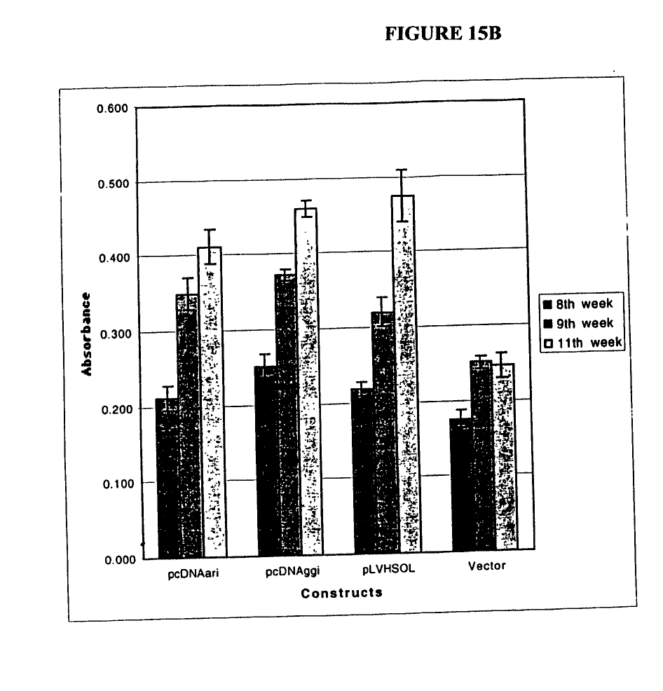

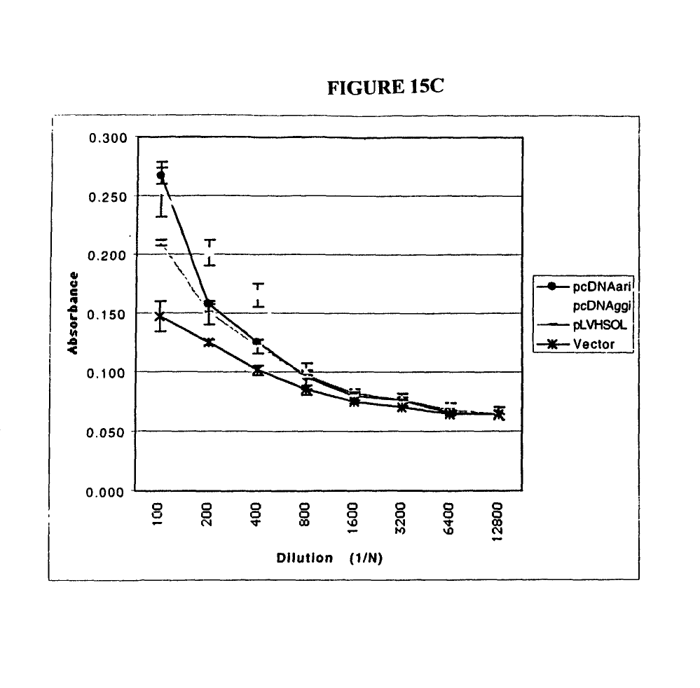

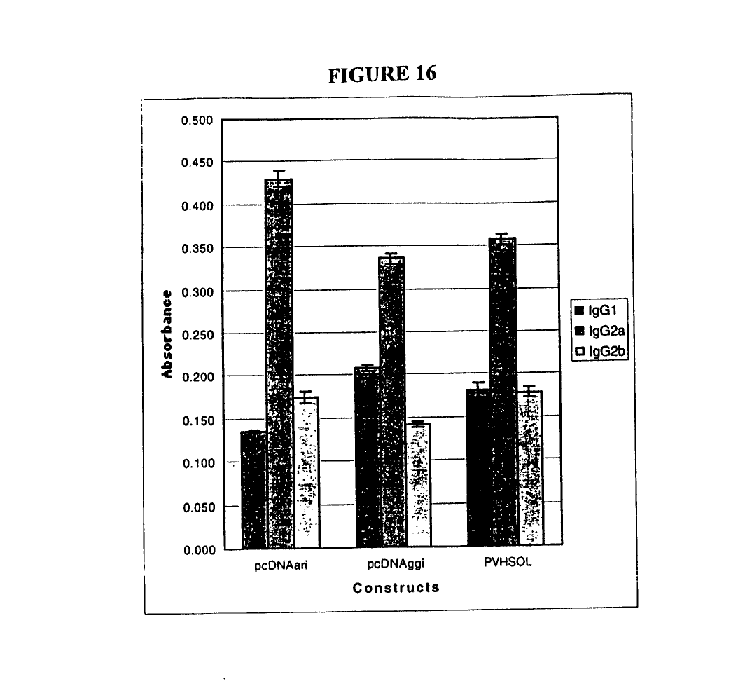

To examine whether DNA vaccination can prime for an augmented carbohydrate response, DNA-minigen vaccinated mice were boosted with 40 ug of polyvalent synthetic LeY with QS-21 on week 8 and bled on week 9 and 11. Serum was diluted to 1:50 and tested for LeY reactivity by ELISA. The results shown in FIG. 15A (IgM) and 15 B (IgG) suggest that the DNA-minigen immunization induced the formation of LeY specific memory cells that were rapidly and specifically stimulated upon encounter with LeY. As expected, boosting with LeY leads to an enhanced anti-LeY IgM response. IgM levels increased a week after immunization and the IgM levels dropped two weeks after the immunization. The anti-LeY IgG antibody response in the DNA vaccinated mice boosted with LeY is significantly higher than with vector alone primed/LeY boosted mice. Importantly, the level of IgG continued to increase in all minigen immunized mice. The IgG titer following this boost increased to a 1:800 titer compared to vector alone primed/LeY-boosted mice as shown in FIG. 15C. We also determined the isotype components at week 11 (FIG. 16). We observed a similar isotype pattern as that prior to the boost. This confirms that we have converted the isotype of the induced anti-carbohydrate response to a stronger and more preferred Thl type response. We continued to monitor the IgG response in boosted mice, the IgG level in two mice out of four in each group continued to rise over the period of the study (not shown).

T helper cells play a crucial role in magnitude and direction of immune responses. T cell proliferation was studied as an indicator of CD4+T-cell activation. Several relevant MAP (106, 611 and 612) were used to stimulate T cells. We did not observe any significant differences between immunized groups in T-cell proliferation index. The data do not clarify the advantage of having included a T helper epitope, however the pcDNAggi plasmid showed a significantly better antibody response.

Enhancement of T Cell Proliferation with Cytokine cDNAs

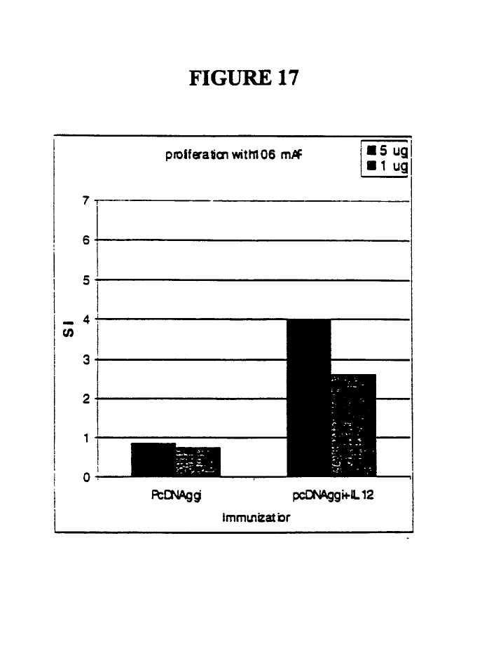

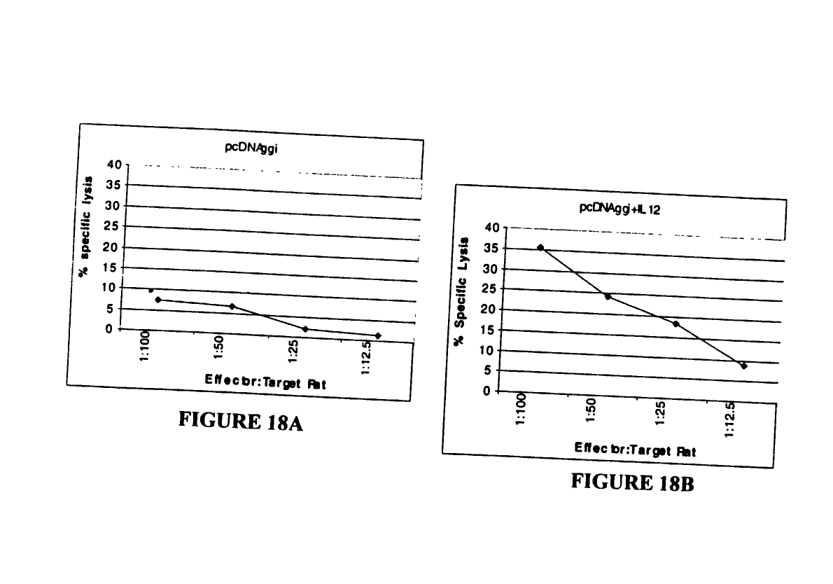

To further study if augmentation of T helper activity will affect the direction and magnitude of the immune response, we co-immunized other groups of mice with the plasmids in FIG. 1 along with plasmids encoding IL-4 and IL-12. IL-12 and IL-4 were selected as representative cytokines associated with Th type 1 and type 2 responses, respectively. Co-immunization with either cytokine encoding plasmids did not influence any further the antibody response to LeY relative to that previously shown with peptide encoded plasmid alone. However, we observed a significantly higher T cell proliferation of splenocytes derived from mice immunized with the pcDNAggi plasmid with co-immunization with IL-12 (FIG. 17). We did not observe any significant T cell proliferation with peptide encoded plasmid alone. Detected INF-γ production (now shown) in collected supernatant of in vitro peptide activated splenocytes indicated a Th1 type activation consistent with the IgG2a production. Expectedly, the Th type 1 response leads to a high cytotoxic T lymphocyte activity against peptide-pulsed P815 target cells (FIGS. 18A and 18B). We could not detect any enhancement in T-cell proliferation or CTL and antibody response upon co-immunization with IL-4.

Discussion

Carbohydrates are traditionally viewed as T cell-independent antigens with a number of unique and important immunological properties that are not encountered when inducing an immune response to proteins. These properties include no overt requirement for the presence of T cells to induce an immune response, dominance of IgM, failure to induce memory following immunization, an absence of affinity maturation following immunization, poor immunogenicity in infants, the elderly and the immunocompromised, and an inability to provide maternal protective antibodies. These properties have precluded the use of pure carbohydrate or polysaccharide vaccines in those patients most at risk.

Conjugate vaccine technology, where a carbohydrate antigen is coupled chemically to a protein carrier, has overcome some of the limitations of carbohydrates as vaccine antigens by rendering the carbohydrate moiety of such vaccines immunogenic, even in the very young. However, based on early studies of carbohydrate conjugates it appears than an immune response to a thymus-dependent form of polysaccharides requires the presence of Lyb-5+ lymphocytres. Lyb-5+ cells are required for an anti-polysaccharide response even when the immunizing antigen is thymus-dependent. Consequently, immunization of xid mice with PS-conjugates seldom results in an anti-PS response indicating that whatever T cell help is provided by an immunologic carrier protein, it is not carbohydrate specific.

To further augment anti-carbohydrate immune responses we have been developing peptide mimics of carbohydrate antigens. Immunizations with peptide mimotopes also have limitations. The ability to induce an anti-carbohydrate response with a Th1 associated antibody response (IgG2a) has very important implications in vaccine development against a variety of difficult target pathogens, as well as a possibly important role in tumor immunotherapy. In an effort to further skew the cross-reactive immune response to a more Th1 like response and to take direct advantage of the in vivo cellular response, we have encoded mimotopes into plasmids using a DNA vaccination approach. DNA immunization has been used successfully to induce both humoral and cellular immunity to a wide variety of pathogens, used to prevent or inhibit tumor development, and to inhibit IgE responses by diverting the immune response from Th2 to Th1 helper cell dominance.

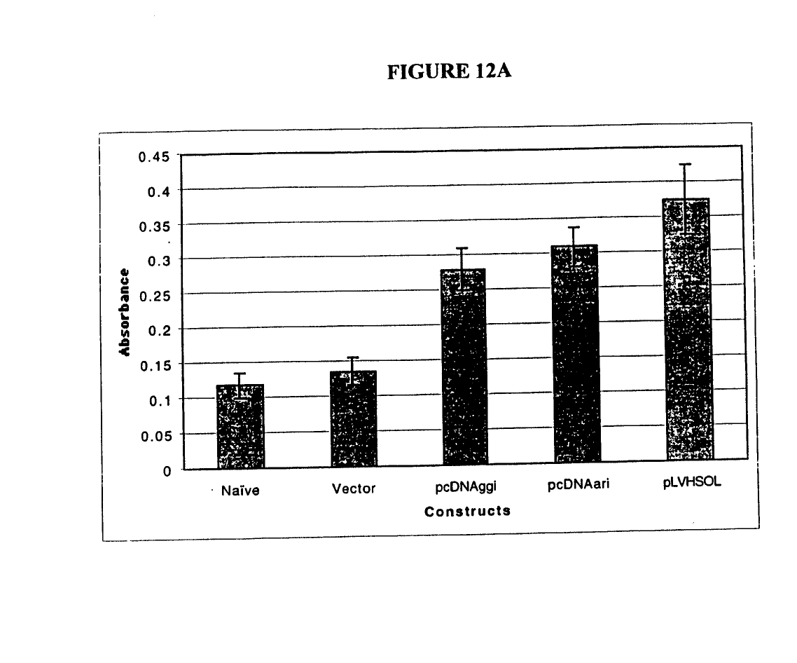

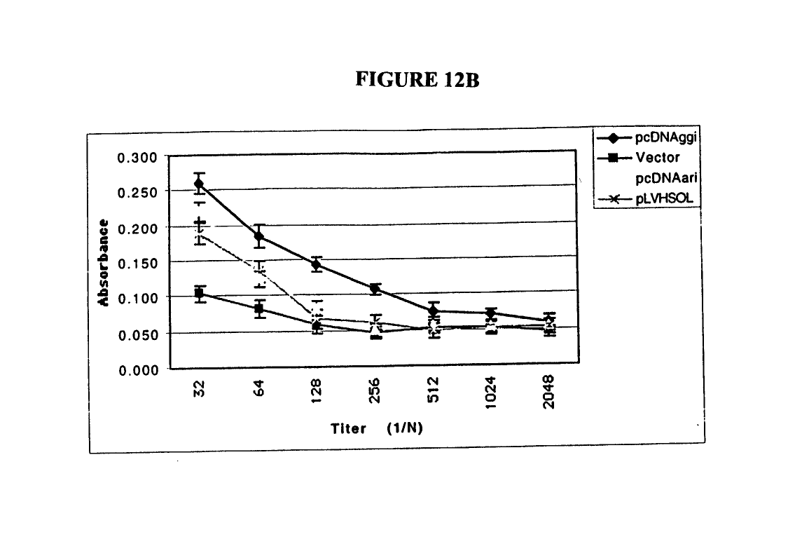

Our data clearly show that we can substitute peptide immunization for DNA immunization. Serum reactivity with LeY after 3 weeks (FIG. 12A) show that we could induce a strong cross-reactive immune response to LeY antigen using several different designed constructs. Comparing the magnitude of the immune response in pcDNAari and pVHSOL immunized mice indicates that using an additive T helper epitope did not increase the primary antibody response. One reason for the lack of more significant difference between these two constructs could be due to the fact that the peptide engineered in the pVHsol epitope, when delivered as a DNA immunogen, has instrinsic T helper capability as predicted (Table 5). This hypothesis is consistent with our results. Immunization with pcDNAggi induced a consistently growing anti-LeY response that appears superior to the other two plasmids. The mimotope in pcDNAggi reflects a repetitive antigen which are typically more immunogenic.

DNA vaccines induce a Thl response with a predominance of IgG2a isotype. It is likely that plasmids are effective adjuvants in providing amplification of IgG2a responses. IgG2a and IgG3 isotype have been reported to be particularly effective in conferring protection against encapsulated organisms and Thl responses are associated with more efficacious tumor vaccination. Our isotyping results (FIG. 13) indicate an anti-LeY IgG2a dominant response compared to IgG1, which is indicative of a Th1 immune response. The results are in agreement with some other reports, where a predominant IgG2a was generated following immunization with DNA encoding influenza NP or hemagglutinin (AA), HIV, HSV, and others.

DNA priming and protein boosting has been used by several groups to enhance the production and functionality of induced antibody. Here, we tried the boost strategy with mimicked carbohydrate. Our results after boost (FIGS. 15A, 15B, 15C) indicate the potential use of a DNA prime and carbohydrate boost for enhancement of antibody response. We induced a rapid and temporary IgM response that is commonly observed following carbohydrate immunization. Despite this temporary IgM response, enhancement of IgG was persistent with a titer three times higher than the pre-boost levels (FIG. 15C). The IgG level slightly increased up to 5 weeks after carbohydrate boost. Most importantly, IgG2a remained the predominant component of the immune response (FIG. 16). This finding suggests that an IgG2a polysaccharide antibody deficiency might be overcome by priming with DNA encoded peptide mimotope followed by a booster with a polyvalent polysaccharide vaccine.

The ability of IL-12 to dramatically enhance TI Ab responses suggests that IL-12 will be useful as a powerful vaccine adjuvant to induce protective immune responses against encapsulated pathogens. By co-injection with IL-12 enclosing plasmids, we obviously could increase the T helper response (FIG. 17), but we did not see any significant increase in the level of antibody production. It seems that the increase in helper function mostly affects the cellular response rather than the humoral response. This conclusion is evident by the observed CTL activity directed to peptide-pulsed p815 cells as targets (FIG. 18A and 18B). As in other studies, IL-12 appears to act as a therapeutic adjuvant to enhance Ag cDNA-induced protective immunity by induction of Th1-associated immune responses. A dynamic interplay between Th1 and Th2 cytokines is responsible for net IFN-γ production. The ability of exogenous IL-12 to strongly shift this balance toward Th1 provides an immunological basis for using it in conjunction with peptide mimotope encoded DNA vaccination for cancer immunotherapy as well as for vaccine development.

Methods

Construction of the Expression Vectors:

For the construction of expression vectors coding for peptides of interest in pcDNAari and pcDNAggi, oligonucleotides were senthesized and inserted into pcDNA3 vector (invotrogen, San Diego, Calif.) fused in frame to an oligonucleotide coding for adenovirus E3 leader sequence. We took advantage of a T helper epitope, so the oligonucleotides for peptides designed in a way that we have T helper epitope in the beginning of peptide sequence. PcDNAggi was generated by inserting of the

| | |
|---|---|
| AGCTTCCACCATGAGGTACATGATTTTAGGCTTGCTCGCCCTTGCGGCAGTCTGCAGCGC | (SEQ ID NO:62) |

oligonucleotide between the restriction sites HindIII and NotI. Then,

| | |
|---|---|
| GGCCGCGAAGCAGATCATCAACATGTGGCAGGAGGTGGGCAAGGCCATGTAC | (SEQ ID NO:63) |
| GCCGGCGGCATCTACTGGCGCTACGACATCTACTGGCGCTACGACATCTACTG | |
| GAGGTACGACTAAT | |

was further cloned into the NotI and XbaI sites. The later oligonucleotide was replaced by the following one to generate the pcDNAari plasmid.

| | |
|---|---|
| GGCCGCGAAGCAGATCATCAACATGTGGCAGGAGGTGGGCAAGGCCATGTAC | (SEQ ID NO:64) |
| GCCCGCATCTACTACCGCTACGACGGCTTCGCCTACTAAT | |

All inserts were sequenced after construction. Plasmids were grown in Escherichia coli DH5a strain. DNA was purified using a maxi prep kit (QIAGEN, Valencia, Calif.). PVHSOL plasmid was generated in the same way as pcDNAari but without a T helper epitope encoding sequence.

DNA immunization

A facilitated DNA inoculation protocol which results in increased in vivo protein expression levels from plasmid delivered genes in vivo was used to immunize mice. Each Balb/c mouse (group of 4) received two intramuscular injections (three weeks apart) with 100 μg of each DNA construct re-suspended in 100 μl of phosphate buffered saline (PBS) and 0.25% bupivacaine-HCl (Sigma, St. Louis, Mo.).

ELISA Assays

Sera from each group of mice were collected and pooled. To assess anti-carbohydrate reactivity of sera collected Standard ELISA was performed. Briefly, wells were coated at 4° C. overnight with 2ug/well and 0.2ug/well of peptide and carbohydrate respectively. After blocking with 0.5% FCS in PBS serum samples were added to each well in triplicate. The plates were then incubated at 37° C. for two hours. Goat anti-mouse IgG conjugated with horseradish peroxidase (Sigma) was then used at 1:10000 dilution with 1 -hour incubation at 37° C. As substrate, we used TMB (Sigma) in citrate buffer and the plates were read at 450 nm. For isotype detection, we used Immunopure® Monoclonal Antibody Isotyping Kit I (Pierce, Rockford, Ill.) based on the manufacturer instruction.

T-cell Proliferation

Splenocytes from experimental and control animals were used for detection of T-cell proliferation as described. Briefly, cells were incubated without or with MAP peptides and after 3 days of incubation 1 μCi of ³H-thymidine was added to each well for 16-18 h. Then cells were harvested using Tomec Mach II 96 cell harvester and counted on a Betaplate liquid scintillation counter (Wallac, Inc.).

Cytotoxic T lymphocyte assay

Cytotoxic activity was measured by a standard 5 hours Cr-51 release assay. Briefly, the effector cells were stimulated six days in the presence of stimulator cells and 10% RAT-T-STIM without Con A (Becton Dickinson Labware, Bedford, Mass.). Peptide pulsed p815 cells were used as stimulators (10:1 effector: stimulator ratio) and targets. Target cells were labeled with 100 μCi/ml Na₂⁵¹ CrO₄ and mixed with effector cells at effector: target (E: T) ratios ranging from 100:1 to 12:1. Maximum and minimum (spontaneous) release was determined by lysis of target cells in 10% Triton X-100 and medium, respectively. Specific lysis was calculated as {(Experimental-spontaneous)/(Maximum-spontaneous)}×100. An assay was not considered valid if the value for the “spontaneous release” counts were more than 20% of the “maximum release.” To calculate specific lysis of targets, the percent lysis of non-specific targets (p815 cells) was subtracted from the percent lysis of specific targets (pulsed p815 cells).

TABLE 5

Plasmids used in this study

| | | | | |
|---|---|---|---|---|
| Plasmid | | | | Peptide |
| Designation | Vector | Leader Sequence | T cell Sequence | Sequence |

| | | | | | |
|---|---|---|---|---|---|
| pcDNAari | pCDNA3 | MRYMILGLLALAAVCSAA | SEQ ID NO:65 | KQIINMWQEVGKAMYA (36) | ARIYYRYDGFAY (60) |
| | | | | SEQ ID NO:66 | SEQ ID NO:67 |
| pcDNAggi | pCDNA3 | MRYMILGLLALAAVCSAA | SEQ ID NO:65 | KQIINMWQEVGKAMYA (34.5) | GGIYWRYDIYWRYDIY (2000) |
| | | | | SEQ ID NO:66 | SEQ ID NO:68 |
| pVHSOL | pBBKL | | | | ARIYYRYDGFAY (60) |
| | | | | | SEQ ID NO:67 |

* Values in parenthesis are estimated half time of disassociation of a molecule containing this sequence using a subsequence of 10 for scoring.

### Example 10

Induction of Carbohydrate Cross-Reactive Immune Responses Upon Immunization with Peptide Mimotope Minigen Encoded Plasmid Vectors

### Abstract

Polysaccharides associated with encapsulated bacteria, viruses and some tumors are weakly immunogenic due to their T cell-independent nature. Even when converted to T cell-dependent forms through conjugation to immunologic carrier proteins, polysaccharide-conjugates induce responses that are deficient in many respects that include the lack of induction of T helper type 1 (Th1) responses that are associated with cellular responses.

We have shown that immunization with peptide mimotopes of nominal carbohydrate antigens elicit cross-reactive antibody responses to tumor cells and pathogens, that include HIV-1, that express the mimicked carbohydrate antigen(s). In an attempt to set strategies for the design and development of effective anti-carbohydrate vaccines, we converted peptide mimotopes of the neolactoseries antigen Lewis Y (LeY) to a DNA sequence (minigen), cloned into an expression vector. Here, we show that immunization with peptide mimotope-encoded plasmids mediate a Th1 response, inducing LeY cross-reactive IgG2a antibodies. The IgG2a immune response persists for at least 2 months. Immunization induces a LeY specific memory response that is rapidly activated upon encounter with nominal antigen upon subsequent boost. Results also show a significant HIV-1 anti-gp120 IgG response after immunization with one of the designed minigens.

Introduction

Most carbohydrate antigens belong to the category of T-cell independent (T1) antigens that reflect their inability to simulate MHC class II dependent T cell help. Carbohydrates alone are not capable of inducing secondary immune responses and require extensive adjuvancity, to convert them to T cell dependent antigens.

We have developed peptides that induce immune responses to carbohydrate structures with in vitro and in vivo functionality. We hypothesize that the utility of peptide mimotopes when formulated into a DNA based vaccine (minigen) could potentially increase the breadth of the immune response, redirecting the response to include the induction of Th1 like profile. Targeting polysaccharide of encapsulated bacteria or tumor associated carbohydrate antigens may augment vaccine responsiveness to these otherwise challenging antigens. Here we report on the feasibility of peptide mimotopes constructed into DNA plasmids for the induction of an anti-carbohydrate immune response. The neolactoseries antigen Lewiy Y (LeY) is used as model system. This antigen is expressed on human carcinomas and on HIV-1 envelope protein.

Materials and Methods

Construction of the Expression Vectors:

For the construction of expression vectors coding for peptides of interest in pcDNAari and pcDNAggi, oligonucleotides were synthesized and inserted into pcDNA3 vector (Invitrogen) fused in frame to an oligonucleotide coding for adenovirus E3 leader sequence. We took advantage of a T helper epitope, so the oligonucleotides for peptides designed in a way that we have T helper epitope in the beginning of peptide sequence.

TABLE 6

Plasmids used in their encoded amino acid sequences.

| | | | | |
|---|---|---|---|---|
| Plasmid | | | | |
| Designation | Vector | Leader Sequence | T cell Sequence | Peptide Sequence |

| | | | | | | |
|---|---|---|---|---|---|---|
| pcDNAari | pcDNA3 | MRYMILGLLALAAVCSAA | SEQ ID NO:65 | KQIINMWQEVGKAMYA | SEQ ID NO:66 | ARIYYRYDGFAY |
| | | | | | | SEQ ID NO:67 |
| pcDNAggi | pcDNA3 | MRYMILGLLALAAVCSAA | SEQ ID NO:65 | KQIINMWQEVGKAMYA | SEQ ID NO:66 | GGIYWRYDIYWRYDIY |
| | | | | | | SEQ ID NO:68 |

DNA Immunization

Each Balb/c mouse (group of 4) received two intramuscular injections (three weeks apart) with 100 μg of each DNA construct re-suspended in 100 μl of phosphate buffered saline and 0.25% bupivacaine-HCl (Sigma).

ELISA ASSAYS

Sera from each group of mice were collected and pooled. Wells were coated at 4° C. overnight with 0.2 μg/well of carbohydrate. Blocking was done with 0.5% FCS in PBS. Other procedures were done as standard. For isotype detection we used Immunopure® Monoclonal Antibody Isotyping Kit I (Pierce) based on the manufacturer instruction.

Results

Anti-carbohydrate reactivity:

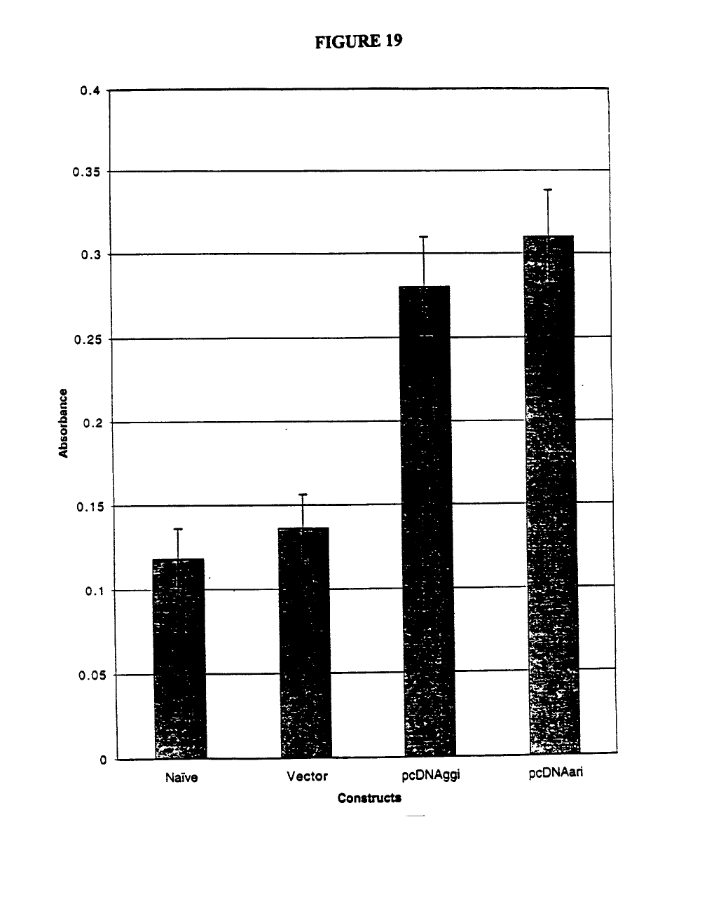

We examined serum reactivity with LeY, by ELISA, 3 weeks after first immunization. Ten times diluted sera showed a significant level of cross-reactive antibody binding after immunization with both constructs (FIG. 19).

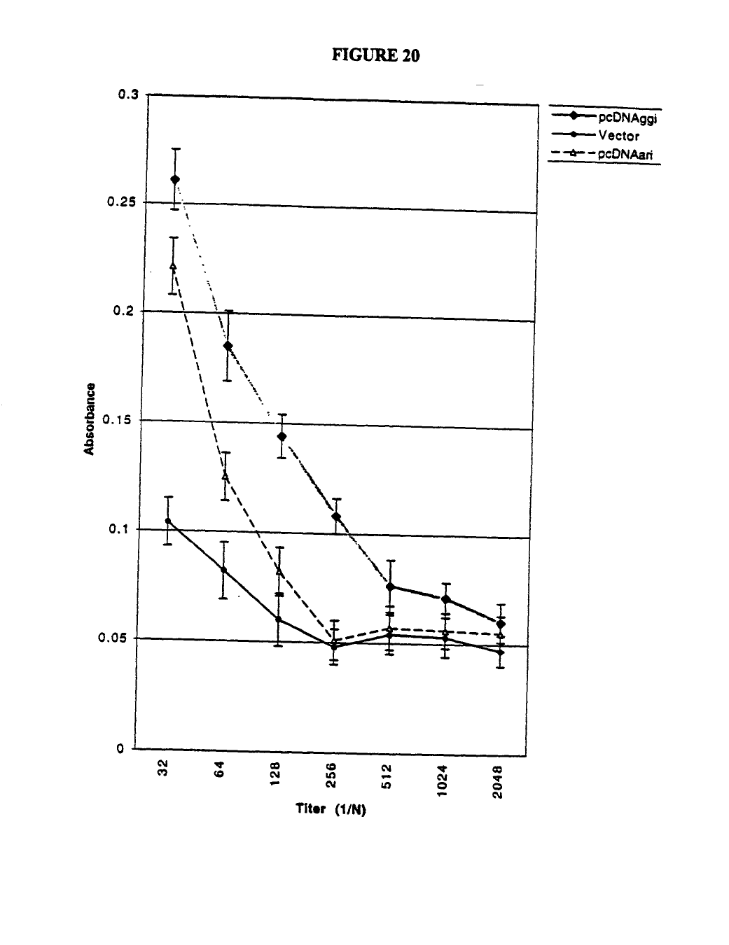

We immunized the mice again at week 3 and determined the titration of the anti-LeY immune response at week 5 (FIG. 20). Results show a cross reactive immune response persistent to a 1:256 dilution in the serum from pcDNAggi immunized animals as compared to vector immunized mice. Immunization with pcDNAggi plasmid induces a higher titer cross-reactive response to LeY in contrast to pcDNAari plasmid.

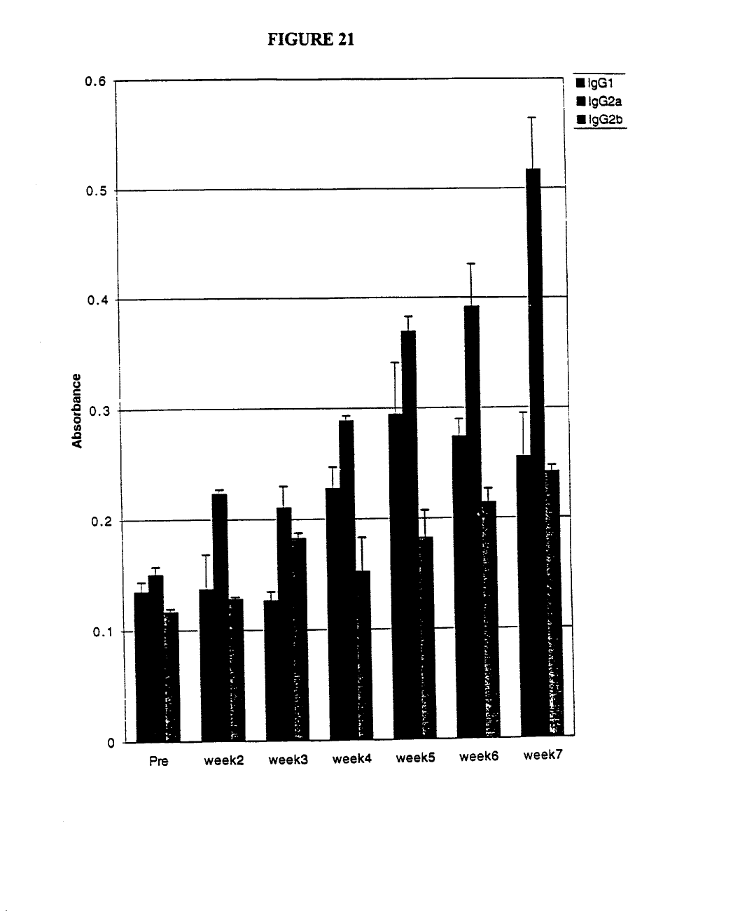

The plasmid pcDNAggi immunized group was selected to further reveal the isotype components of this cross-reactivity. Importantly, the response had been switched to a predominantly IgG2a isotype by week 7 (FIG. 21). This is contrast to polysaccharide or peptide mimotope immunization that induced predominantly the IgM isotype. This switch in isotype is likely the result of the Th 1 bias of the now converted DNA immunogen.

DNA Vaccination Primes for Higher Antibody Response

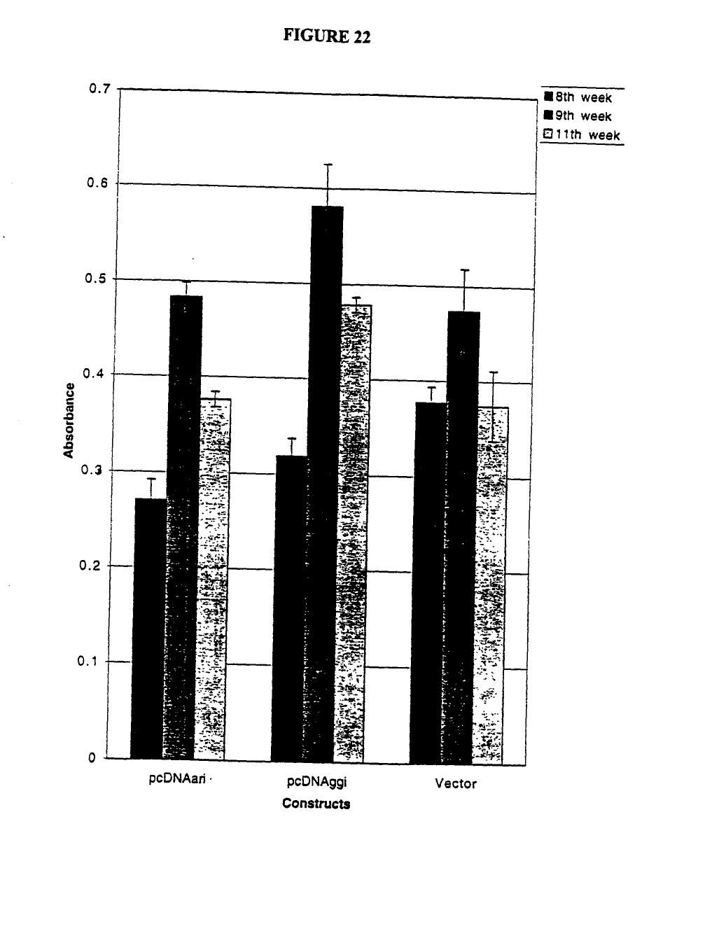

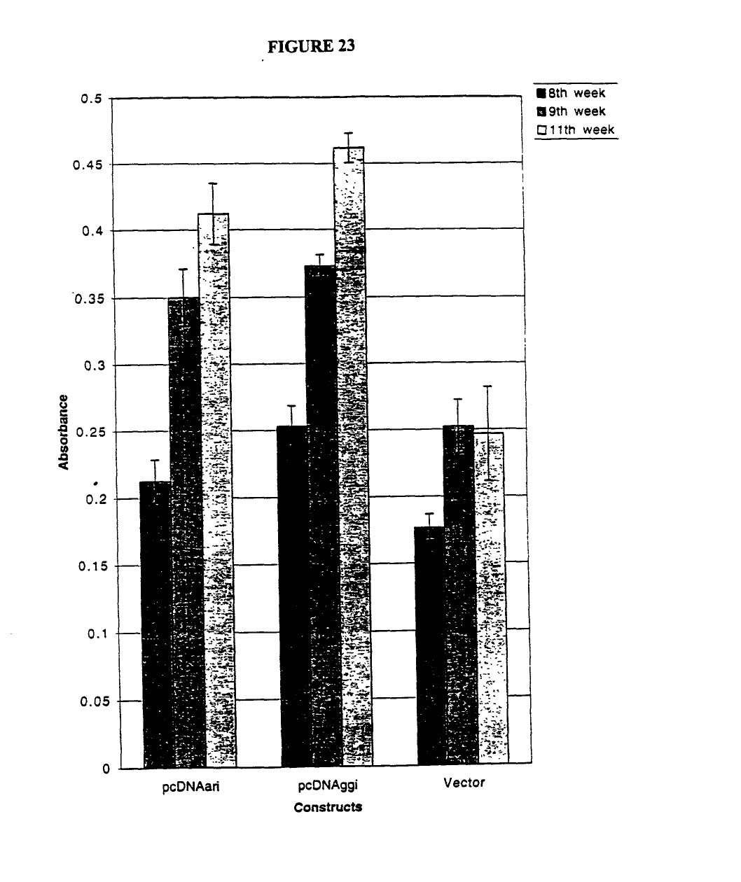

DNA-minigen vaccinated mice were boosted with 40 μg of polyvalent synthetic LeY with QS-21, as adjuvant, on week 8. Mice were bled on weeks 9 and 11. Serum was diluted to 1:50 and tested for LeY reactivity by ELISA. The results shown in FIG. 22 (IgM) and FIG. 23 (IgG) support that the DNA-minigen immunization induced the formation of LeY specific memory cells which were rapidly and specifically stimulated upon encounter with LeY. As expected, boosting with LeY leads to an enhanced anti-LeY IgM response. IgM increased a week after immunization and the IgM levels dropped two weeks after the immunization.

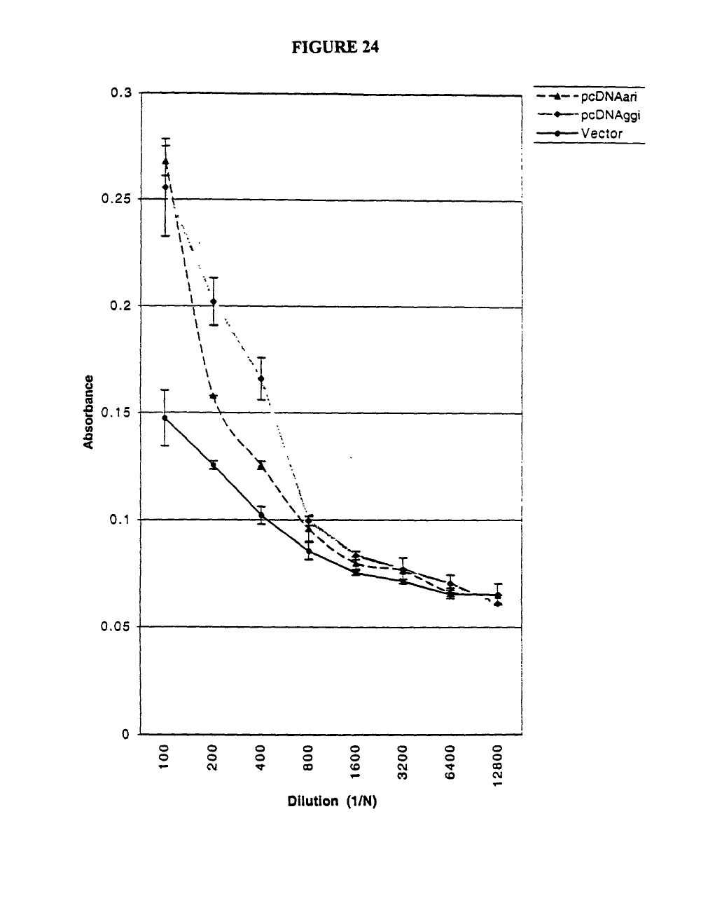

The anti-LeY IgG antibody response in the DNA vaccinated mice boosted with LeY is significantly higher than with vector alone primed/LeY boosted mice. Importantly, the level of IgG continues to increase in both groups of minigen immunized mice. The IgG titer of pcDNAggi immunized mice following this boost increased to a 1:800 titer compared to vector alone primed/LeY boosted mice as shown in FIG. 24.

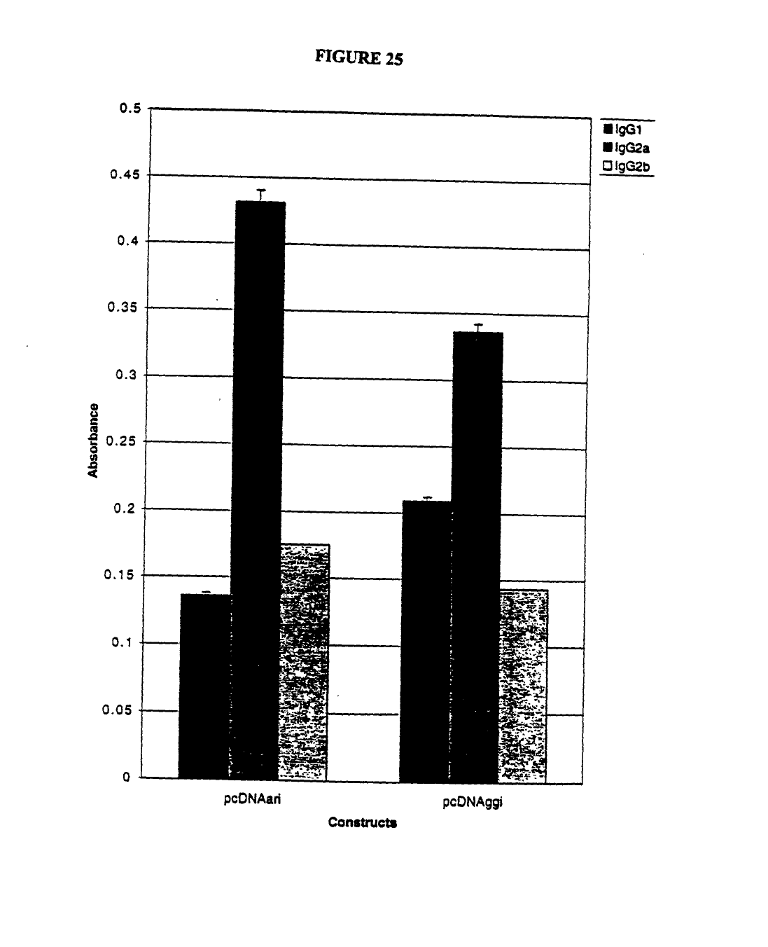

We also determined the isotype components at week 11 (FIG. 25). We observed a similar isotype pattern as we observed prior to the boost. This confirms that we have converted the isotype of the induced anti-carbohydrate response to a stronger and more preferred Thl type response.

Sera from Immunized Mice Bind to Glycosylated gp120

To determine if after immunization there is an antibody response cross-reactive with HIV envelope, collected sera three weeks after the second injection were examined for binding to glycosylated gp120. Sera were diluted 1:50 and their reactions were examined by a standard ELISA.

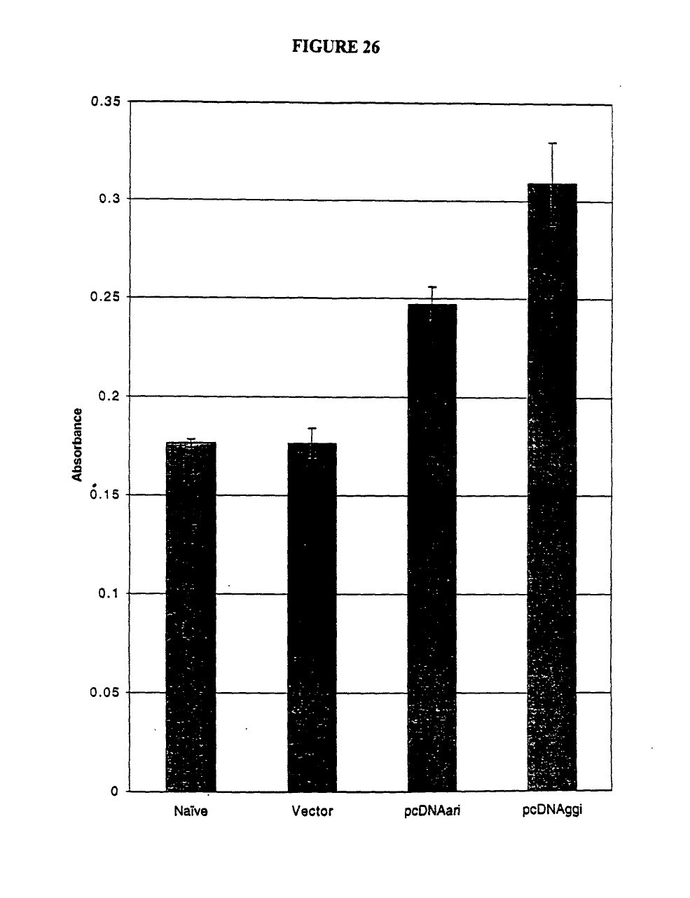

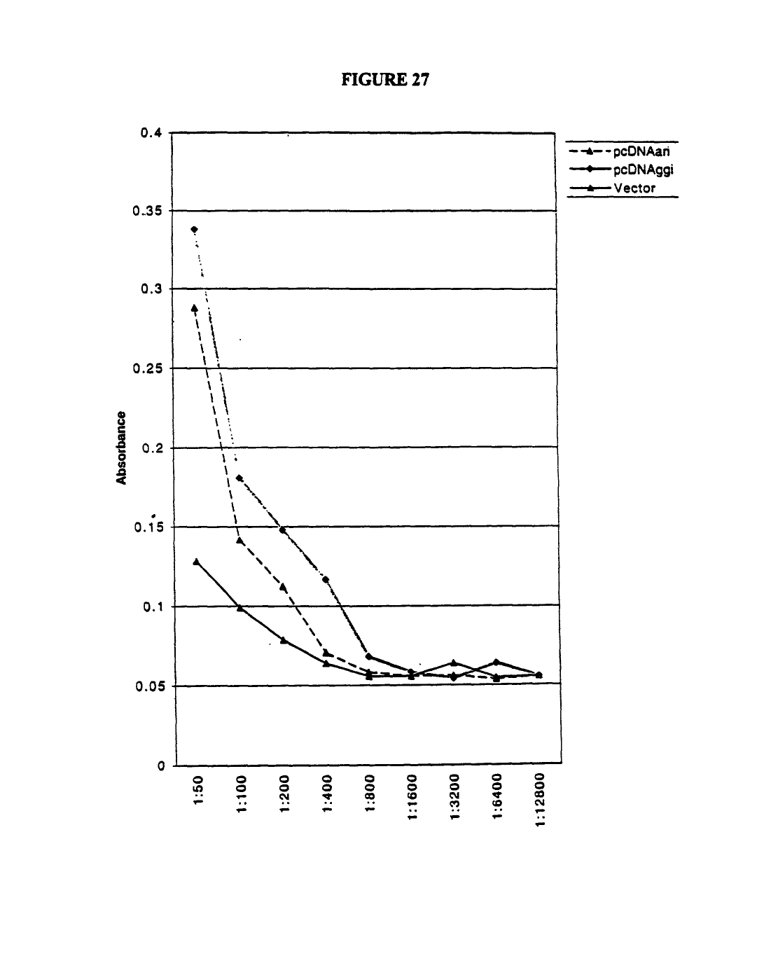

As shown in FIG. 26 both constructs induce a cross reactive antibody response to glycosylated gp120. However, serum from pcDNAggi immunized animals showed higher binding. We checked the antibody titer against glycosylated gp120. Results of a representative experiment out of three are shown in FIG. 27. A significant difference is observed up to 1:400 dilution.

Conclusions

1-Immunization with a DNA vaccine encoding for peptide mimotopes (minigen) induces humoral immune responses.

2-The minigen immunization induces a Th1 response with a predominant IgG2a isotype.

3-Our results indicate the potential use of a DNA prime and carbohydrate boost for enhancement of antibody response.

4-By priming with DNA encoded peptide mimotope followed by a boost with polysaccharide vaccine will overcome IgG2a deficiency.

5-Immunization with a peptide mimotope encoded DNA vaccine, which mimics the LeY structure presented on the surface of HIV-1 gp120, induces anti-gp120 antibody production.

### Example 11

Application areas of this technology include bacterial, viral and cancer vaccines for neonates, cancer patients and other immunocomprised individuals. For example, at the present time, infants receive 14 parenteral immunizations by 18 months of age. Simplication of immunization schedules may be achieved by combining vaccines and by mucosal vaccine administration. Finally, cost associated with immunization must remain low to be economically feasible in developed and developing countries. Guided by these particular requirements for a successful vaccine candidate, we propose to develop DNA vaccines that enhance carbohydrate cross-reactivities. Recent advances in vaccine development have demonstrated the feasibility of inducing protective immunity against various pathogens through the administration of engineered DNA vectors. We hypothesize that administration of a DNA vaccine resulting in endogenous expression of carbohydrate peptide mimics will induce protective immunity against encapsulated organisms, other pathogens and against tumor cells. The advantages associated with DNA-based vaccines include ease of construction and standardization, stability and heat resistance, the ability to incorporate representation of several serogroups in one vector, ability to insert potent immunogenic motifs such as CpG, induction of a T helper cell type 1 (Th1) response and extremely low cost of production. Intranasal immunization will potentially enhance the benefits of this vaccine preparation by facilitating ease of administration, low cost and by stimulating an effective mucosal immune response. This may be particularly effective strategy in infants as the mucosal immunity matures to adult level within the first months of life, in contrast to systemic immunity.

In summary, DNA vaccines can potentially overcome the problems associated with both native polysaccharide as well as the conjugate polysaccharide vaccines. The Th1 nature of the immune response to endogenous generated peptides may allow for effective immunization in infancy and induce long-lasting mucosal and systemic immunity. The inherent stability of this type of vaccine preparation eliminates the need for refrigeration. Decreased nasopharyngeal colonization will prevent spread of bacteria resulting in a decrease of disease incidence, even in the nonresponding or unimmunized population. Peptides representing several serogroups may be cloned into one vector offering a practical and very economical method of inducing protective immunity against pathogens and tumor cells.

TABLE 1
Bacterial LOS Mimic Host Glycosphingollpids

| GSL series type | Structure | Bacterial species |
|---|---|---|
| Lacto | Galβ1→4GlcNAcβ1→3Galβ1→4Glcβ1→Cer | N. gonorrhoeae |
| | | N. meningitidis |
| | | N. lactamica |
| | | N. cinera |
| | | H. influenzae type b(S) |
| | | H. influenzae NT (S) |
| | | H. influenzae biotype aegyptius |
| | | H. ducreyi (S) |
| Globo | Galα1→4Galβ1→4Glcβ1→Cer | N. gonorroheae |
| | | N. meningitidis |
| | | H. influenzae type b |
| | | H. influenzae NT |
| | | B. catarrhalis |
| Gangilo | GalNAcβ1→4Galβ1→4Glcβ1→Cer | N. gonorrhoeae |
| | Galβ1→3GalNAcβ1→4Galβ1→4Glcβ1 | |
| | GalNAcβ1→3Galβ1→4GlcNAcβ1→3Galβ1→4Glcβ1→Cer | |

TABLE 2
Peptide motifs that mimic carbohydrate structures

| | Motif | Carbohydrate | Structure | Reference |
|---|---|---|---|---|
| SEQ ID NO: 4 | YYPY | Mannose | methyl-α-D-mammopyramoside | (54, 55) |
| | WRY | Glucose | α(1-4)glucose | |
| SEQ ID NO: 5 | PWLY | Lewis Y | Fucα1→2Galβ1→4(Fucα1→3)GlcNAc | (56) |
| SEQ ID NO: 7 | YYRYD | GroupC | α(2-9)sistic acid | (44) |
| | | Polysaccharide | | |

TABLE 3
Binding of Various Anti-Peptide Sera to Different Cells As Measured by FACS

| | Anti P1 | Anti P2 | Anti 105 | Anti 107 | ME361 | BR55-2 |
|---|---|---|---|---|---|---|
| Cell Lines | (YYPY) | (YYRYD) | MAP | MAP | (100 μg/ml) | (100 μg/ml) |
| SKBR5 | 60.0 | 86 | ND | ND | 3.1 | 59.0 |
| MCF7 | 63/144* | 54 | 150 | 176 | 5.4 | 352 |
| SKBR3 | 240.6 | 275.6 | 240 | 250 | 3.2 | 235.6 |
| HS578 B51 | 17.8 | 19.9 | 18.6 | 18.4 | ND | 16.2 |
| (normal | | | | | | |
| breast) | | | | | | |
| SKMEL-28 | 47.0 | 33.0 | ND | ND | 26 | 13.8 |
| WM793 | 145.5 | 42.3 | ND | ND | 92.1 | 15.4 |
| NIH3T3 | 20.9 | 21.8 | 24.5 | 21.2 | ND | 15.3 |
| Murine | | | | | | |
| Fibroblasts | | | | | | |

Background flourescence (Mean Fluorescence) associated with non-specific mouse sera is 24.2, and 23.7 for SKBR3, and NIH 3T3 cells, respectively. ME361 is 14.0 and 10.0 for SKBR5 and MCF7. Background for the human melanoma line was on average 24.4. (Final Sera Concentration: 1:50).
*final dilution at 1:20.

TABLE 4
Summary of Complement Dependent Cytotoxicity Results

| Tumor | C1 | P2 | P1 | P3 | G1 | G2 | B1 | LeY-PAA | ME361 | | BR55-2 | |
|---|---|---|---|---|---|---|---|---|---|---|---|---|
| SKME1-28 | 3 | 13 | 10 | 32 | 75 | 87 | 10 | 4 | 53 | (50 μg) | 3 | (100 μg) |
| SKBR3 | 6 | 80 | 90 | 86 | 10 | 13 | 90 | 20 | 10 | (100 μg) | 80 | (100 μg) |
| MCF-7 | 3 | 29 | 66 | 56 | 20 | 15 | 70 | 26 | 5 | (50 μg) | 75 | (100 μg) |
| WM793 | 5 | 9 | 9 | 28 | 90 | 90 | 10 | 2 | 63 | (30 μg) | 1 | (100 μg) |
| OVAR-3 | 5 | 84 | 89 | 86 | 9 | 11 | 85 | 25 | 6 | (50 μg) | 80 | (100 μg) |

Values are averaged percent cytotoxicity. Final dilutions are 1:15 for sera. Monoclonal antibody ME361 and
BR55-2 concentrations are per ml.

| Peptides used in these studies | | | | |
|---|---|---|---|---|
| SEQ ID NO: 37 | G1 | GVVWRYTAPVHLGDG | ME361 phage screen | |
| SEQ ID NO: 38 | G2 | LDVVLAWRDGLSGAS | ME361 phage screen | |
| SEQ ID NO: 39 | P1 | GGIYYPYDIYYPYDIYYPYD | Repeating motif of Con A phage screen | |
| SEQ ID NO: 40 | P2 | GGIYWRYDIYWRYDIYWRYD | Repeating motif from amylase | |
| | | | inhibitor | |
| SEQ ID NO: 41 | P3 | GGIYYRYDIYYRYDIYYRYD | Repeating motif of anti-Id | |
| SEQ ID NO: 42 | P4 | GSSFWRYTTYYDPS | FH-6 phage screen | |
| SEQ ID NO: 43 | B1 | IMILLIFSLLWFGGA | BR55-2 phage screen | |
| SEQ ID NO: 44 | C1 | GDTRYIPALOHGDKK | Irrelevant Control | +TL,43 |

What is claimed is:

1. A method of preparing a peptide which mimics an antigenic carbohydrate comprising the steps of:
a) identifying a peptide sequence which is immunogenically cross reactive with an antigenic carbohydrate; and
b) synthesizing a peptide comprising at least two repeating units of said peptide sequence.

2. The method of claim 1 wherein said peptide sequence is identified by screening a peptide library with an antibody against said antigenic carbohydrate.

3. The method of claim 1 wherein said antigenic carbohydrate is selected from the group consisting of histo-blood group related antigens, gangliosides, glycosphingolipids, lipopolysaccharides and lipooligosaccharides.

4. The method of claim 1 wherein said antigenic carbohydrate is associated with a pathogen.

5. The method of claim 1 wherein said antigenic carbohydrate is associated with a tumor cell.

6. The method of claim 1 wherein said peptide consists of up to 28 amino acid residues, but not limited to 28 amino acids.

7. A method of preparing a recombinant antibody which mimics an antigenic carbohydrate comprising the steps of:
a) identifying a peptide sequence which is immunogenically cross reactive with an antigenic carbohydrate; and
b) synthesizing a recombinant antibody comprising said peptide sequence.

8. The method of claim 7 wherein said peptide sequence is identified by screening a peptide library with an antibody against said antigenic carbohydrate.

9. The method of claim 7 wherein said antigenic carbohydrate is selected from the group consisting of: histo-blood group related antigens, gangliosides, glycosphingolipids, lipopolysaccharides and lipooligosaccharides.

10. The method of claim 7 wherein said antigenic carbohydrate is associated with a pathogen.

11. The method of claim 7 wherein said antigenic carbohydrate is associated with a tumor cell.

12. The method of claim 7 wherein said recombinant antibody comprises a variable region that comprises said peptide sequence.

13. The method of claim 7 wherein said recombinant antibody comprises one or more complementarity determining regions that comprises said peptide sequence.

14. A method of generating an immune response against a pathogen or tumor cell in an individual comprising administering to said individual
a peptide comprising at least two repeat units of a peptide sequence which is immunogenically cross reactive an antigenic carbohydrate; and/or
a recombinant antibody which comprises a peptide sequence which is immunogenically cross reactive an antigenic carbohydrate; and/or
a DNA vaccine, live attenuated vaccine or recombinant vaccine which comprises a nucleic acid sequence that encodes a peptide sequence which is immunogenically cross reactive an antigenic carbohydrate operably linked to regulatory sequences.

15. The method of claim 14 wherein said immune response is generated against a pathogen, and either: said individual is free of infection by said pathogen and said immune response is prophylactic; or said individual is infected by said pathogen and said immune response is therapeutic.

16. The method of claim 14 wherein said immune response is generated against a tumor cell, and either said individual is free of said tumor cell and said immune response is prophylactic; or said individual has a tumor comprising said tumor cell and said immune response is therapeutic.

17. The method of claim 14 wherein said antigenic carbohydrate is associated with a pathogen and said peptide is administered in conjunction with vaccine against said pathogen or said antigenic carbohydrate is associated with a tumor cell and said peptide is administered in conjunction with vaccine against said tumor cell.

18. A method of enhancing binding of anti-antigenic carbohydrate antibodies to said antigenic carbohydrate in an individual comprising administering to said individual anti-antigenic carbohydrate antibodies and a peptide comprising 1-3 repeat units of a peptide sequence which mimics an antigenic carbohydrate.

19. A method of inhibiting binding of a ligand to a receptor which is an antigenic carbohydrate comprising administering to said individual a peptide comprising 1-3 repeat units of a peptide sequence which mimics an antigenic carbohydrate.

20. A method of identifying peptide sequences which can induce an immune response against two or more different pathogens, said method comprising the steps of:
a) identifying a peptide sequence which is immunogenically cross reactive an antigenic carbohydrate which is associated with a pathogen;
b) administering an amount of a peptide comprising said peptide sequence to an animal sufficient to induce an immune response against said peptide; and
c) analyzing said immune response to identify two or more pathogens against which said immune response cross reacts.

21. A composition of peptides or proteins consisting of peptide sequences selected from the group consisting of SEQ ID NOS 1-44 or a peptide or protein comprising two or more repeats of peptide sequences selected from the group consisting of SEQ ID NOS: 1-44.

22. A pharmaceutical composition comprising a composition according to claim 21 and a physiologically acceptable carrier.

23. Recombinant antibodies which comprise peptide sequences which immunogenically cross react with antigenic carbohydrates.
